(19) United States
(12) Patent Application Publication
(10) Pub. No.: US 2026/0207726 A1
(43) Pub. Date: Jul. 23, 2026

(54) MULTIVALENT VACCINE COMPOSITIONS AND USES THEREOF

(71) Applicants: Janssen Pharmaceuticals, Inc., Titusville, NJ (US); GlaxoSmithKline Biologicals S.A., Rixensart (BE)

(72) Inventors: Jeroen GEURTSEN, Leiden (NL); Kellen Cristhina FAE, Leiden (NL); Micha Andres HAEUPTLE, Rixensart (BE)

(21) Appl. No.: 19/129,220

(22) PCT Filed: Nov. 14, 2023
(86) PCT No.: PCT/US2023/079642

§ 371 Date: May 12, 2025

Related U.S. Application Data
(60) Provisional application No. 63/383,841, filed on Nov. 15, 2022.

(51) Int. Cl. A61K 39/108; A61K 9/00; A61K 39/00; A61K 39/385; A61P 31/04; C12N 9/90; C12P 19/04; C12P 21/00

(52) U.S. Cl. CPC A61K 39/0258; A61K 9/0019; A61K 39/385; A61P 31/04; C12N 9/90; C12P 19/04; C12P 21/00; C12Y 501/03002; A61K 2039/54; A61K 2039/545; A61K 2039/55583; A61K 2039/575; A61K 2039/62; A61K 2039/70

(57) ABSTRACT

Compositions and methods are described for inducing an immune response against extra-intestinal pathogenic Escherichia coli (ExPEC) to thereby provide immune protection against diseases associated with ExPEC. In particular, compositions are described comprising conjugates of E. coli polysaccharide antigen O1, O2, O4, O6, O8, O15, O16, O18, O25, and O75 and further comprising O153 or O21 or both O153 and O21 covalently bound to a carrier protein for the prevention of invasive ExPEC disease.

### FIELD OF THE INVENTION

This invention relates to the fields of immunology and vaccines. In particular, the invention relates to a composition comprising E. coli O1, O2, O4, O6, O8, O15, O16, O18, O25, and O75 antigen polysaccharides and further comprising O153 or O21 or both O153 and O21 antigen polysaccharides. The invention further relates to the use of such compositions for inducing an immune response against E. coli for the prevention or treatment of E. coli infections, in particular to prevent extra-intestinal pathogenic E. coli (ExPEC) disease.

### CROSS-REFERENCE TO RELATED APPLICATIONS

The present application claims priority to U.S. Provisional Application No. 63/383,841 filed on Nov. 15, 2022, the disclosure of which is incorporated herein by reference in its entirety.

### REFERENCE TO SEQUENCE LISTING SUBMITTED ELECTRONICALLY

The contents of the electronic sequence listing (O04852_206WO1.xml; Size: 30,323 bytes; and Date of Creation: Nov. 13, 2023) is herein incorporated by reference in its entirety.

### BACKGROUND OF THE INVENTION

Extraintestinal pathogenic Escherichia coli (ExPEC) are normally harmless inhabitants of human gut, alongside commensal E. coli strains. However, ExPEC strains can possess virulence factors for the colonization and infection of sites outside of the gastrointestinal tract to cause diverse and serious invasive diseases, resulting in significant morbidity, mortality, and costs annually. ExPEC strains are the most common cause of urinary tract infection (UTI). They are also a contributor to surgical site infections and neonatal meningitis, associated with abdominal and pelvic infections and nosocomial pneumonia, and are occasionally involved in other extra-intestinal infections such as osteomyelitis, cellulitis, and wound infections. All these primary sites of infection can result in ExPEC bacteremia (Russo et al., 2003). Neonates, the elderly, and immunocompromised patients are particularly susceptible to ExPEC infection, including invasive ExPEC disease (IED).

Bacterial resistance to antibiotics is a major concern in the fight against bacterial infection, and multi-drug resistant (MDR) E. coli strains are becoming more and more prevalent.

The O-antigen serotype is based on the chemical structure of the O polysaccharide antigen, the outer membrane portion of the lipopolysaccharide (LPS) in a Gram-negative bacterium. More than 180 serologically unique E. coli O-antigens have been reported (Stenutz et al., FEMS Microbial Rev. 2006; 30:382-403), although the vast majority of ExPEC isolates are classified within less than 20 O-antigen serotypes. Full-length E. coli O-antigens are typically comprised of about 10 to 25 repeating sugar units attached to the highly conserved LPS core structure, with each component synthesized separately by enzymes encoded predominantly in the rfb and rfa gene clusters, respectively. Following polymerization of the O-antigen, the O-antigen polysaccharide backbone may be modified, typically through the addition of acetyl or glucose residues. These modifications effectively increase serotype diversity by creating antigenically distinct serotypes that share a common polysaccharide backbone, but differ in side branches.

ExPEC infection can be caused by any serotype. Although there is an overrepresentation of certain serotypes in ExPEC infection, surface polysaccharides from ExPEC isolates nonetheless exhibit considerable antigenic diversity, which makes the development of an ExPEC vaccine based on surface polysaccharides challenging (Russo et al., Vaccine. 2007; 25:3859-3870). Also, certain O-antigens may be poorly immunogenic. Furthermore, based on studies from Pneumococcal conjugate vaccines, when a number of serotypes can cause a disease, the vaccine composition, such as the choice of serotypes for inclusion in a vaccine and the dosage levels of the included serotypes, can be critical, since use of a vaccine against certain serotypes may potentially increase carriage of and disease from serotypes not included in the vaccine, or even a serotype that is included in the vaccine but only weakly effective in immunizing against the serotype (Lipsitch, Emerging Infectious Diseases; 1999, 5:336-345). Ideally, a vaccine should maximize its beneficial effects in the prevention of disease caused by serotypes included in the vaccine, while minimizing the risk of added disease from increased carriage of non-vaccine serotypes.

Efforts toward the development of a vaccine to prevent ExPEC infections have focused on O-antigen polysaccharide conjugates. A 12-valent O-antigen conjugate vaccine was synthesized through extraction and purification of O-antigen polysaccharide and chemical conjugation to detoxified Pseudomonas aeruginosa exotoxin A and tested for safety and immunogenicity in a Phase 1 clinical study (Cross et al., J. Infect. Dis. (1994) v. 170, pp. 834-40). This candidate vaccine was never licensed for clinical use. A bioconjugation system in E. coli has been developed recently, in which the polysaccharide antigen and the carrier protein are both synthesized in vivo and subsequently conjugated in vivo through the activities of the oligosaccharyl transferase PglB, a Campylobacter jejuni enzyme, expressed in E. coli (Wacker et al., Proc. Nat. Acad. Sci. (2006) v. 103, pp. 7088-93). This N-linked protein glycosylation system is capable of the transfer of diverse polysaccharides to a carrier protein within the bacteria.

Bioconjugation has been used successfully to produce conjugate polysaccharide for an E. coli four-valent O-antigen candidate vaccine (Poolman and Wacker, J. Infect. Dis. (2016) v. 213 (1), pp. 6-13). Ten-valent and nine-valent vaccine compositions of bioconjugates of E. coli O-antigen polysaccharides were previously described (e.g. WO 2020/191082, WO 2022/058945). The development of a successful ExPEC vaccine requires coverage of predominant serotypes, and the presence of further O-antigen modifications in subsets of ExPEC isolates presents a further challenge in covering isolates displaying unmodified and modified LPS. Moreover, immune responses to vaccine compositions comprising O-antigens from multiple serotypes may differ between the serotypes. Accordingly, there is a continued need in the art for vaccines against ExPEC. In particular, there exists a need for an ExPEC vaccine based on surface polysaccharides that can be safely and efficiently manufactured at industrial scale and that can be used to provide an effective immune response, and preferably protection, against ExPEC O153 serotype and/or O21 serotype, and preferably also against other serotypes prevalent among ExPEC disease isolates.

### SUMMARY OF THE INVENTION

In a first aspect, the invention relates to a composition comprising E. coli O1, O2, O4, O6, O8, O15, O16, O18, O25, and O75 antigen polysaccharides and wherein the composition further comprises O21 antigen polysaccharide or O153 antigen polysaccharide or O21 and O153 antigen polysaccharides, wherein each of the antigen polysaccharides is independently covalently linked to a carrier protein. In certain embodiments, the O1 antigen is O1A, the O4 is glucosylated (O4A), the O6 antigen is O6A, the O18 antigen is O18A, and the O25 antigen is O25B. In certain embodiments, the antigen polysaccharides of the composition of the invention comprise the structures as described in Table 1 herein.

In certain embodiments, the composition of the invention further comprises at least one additional E. coli antigen polysaccharide covalently linked to a carrier protein.

In certain embodiments, the E. coli O antigen polysaccharides present in a composition of the invention consist of the: (i) O1, O2, O4, O6, O8, O15, O16, O18, O25, O75 and O153, or (ii) O1, O2, O4, O6, O8, O15, O16, O18, O25, O75 and O21; or (iii) O1, O2, O4, O6, O8, O15, O16, O18, O25, O75, O153 and O21.

In certain embodiments, the carrier protein is detoxified exotoxin A of Pseudomonas aeruginosa (EPA) or CRM₁₉₇. Preferably, the carrier protein is EPA. In certain embodiments, the carrier protein comprises 1 to 20 glycosylation consensus sequences having the amino acid sequence Asn-X-Ser(Thr) wherein X can be any amino acid except Pro, preferably the glycosylation consensus sequences having the amino acid sequence of SEQ ID NO: 1. In certain embodiments, each carrier protein comprises the amino acid sequence of SEQ ID NO: 2.

In certain embodiments, the E. coli antigen polysaccharides of the composition are covalently linked to the carrier protein by bioconjugation or by chemical conjugation. Preferably, the E. coli antigen polysaccharides are covalently linked to the carrier protein by bioconjugation. In certain embodiments, the E. coli antigen polysaccharides are covalently linked to an Asn residue in a glycosylation site in the carrier protein.

In a second aspect, the invention provides a pharmaceutical composition comprising a pharmaceutically acceptable excipient and the composition as described herein.

In a third aspect, the invention provides a method of inducing an immune response to E. coli, preferably extra-intestinal pathogenic E. coli (ExPEC), in a subject, comprising administering to the subject the composition as described herein or the pharmaceutical composition as described herein. In certain embodiments, the method of inducing an immune response to E. coli limits the severity of or prevents an invasive ExPEC disease in the subject, preferably wherein the invasive ExPEC disease comprises sepsis and/or bacteremia.

In a fourth aspect, the invention provides a recombinant prokaryotic host cell for preparing a bioconjugate of an E. coli O153 antigen polysaccharide covalently linked to a carrier protein, the recombinant prokaryotic host cell comprising:
a. a nucleotide sequence of an rfb gene cluster for the O153 antigen polysaccharide;
b. a nucleotide sequence encoding the carrier protein comprising at least one glycosylation site comprising a glycosylation consensus sequence having sequence Asn-X-Ser(Thr) wherein X can be any amino acid except Pro, preferably having SEQ ID NO: 1; and
c. a nucleotide sequence encoding an oligosaccharyl transferase PglB.

In certain embodiments, the E. coli O153 antigen polysaccharide comprises the structure of Formula (O153):

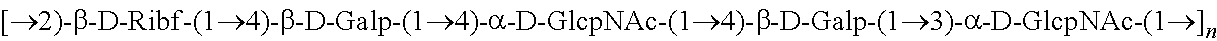

wherein each n is independently an integer of 1 to 40, preferably 5 to 30, preferably 7 to 25.

In certain embodiments, the PglB in prokaryotic host cell for preparing a bioconjugate of an E. coli O153 comprises the amino acid mutations N311V, K482R, D483H, and A669V relative to wild-type PglB having the amino acid sequence of SEQ ID NO: 4. The invention also comprises a method of preparing a bioconjugate of an E. coli O153 antigen polysaccharide covalently linked to a carrier protein, the method comprising culturing the recombinant prokaryotic host cell as described herein to produce the bioconjugate

In a fifth aspect, the invention provides a recombinant prokaryotic host cell for preparing a bioconjugate of an E. coli O21 antigen polysaccharide covalently linked to a carrier protein, the recombinant prokaryotic host cell comprising:
a. a nucleotide sequence of an rfb gene cluster for the O21 antigen polysaccharide;
b. a nucleotide sequence encoding the carrier protein comprising at least one glycosylation site comprising a glycosylation consensus sequence having sequence Asn-X-Ser(Thr) wherein X can be any amino acid except Pro, preferably having SEQ ID NO: 1;
c. a nucleotide sequence encoding an oligosaccharyl transferase PglB; and
d. a nucleotide sequence encoding an UDP-glucose 4-epimerase.

In certain embodiments, the E. coli O21 antigen polysaccharide comprises the structure of Formula (O21):

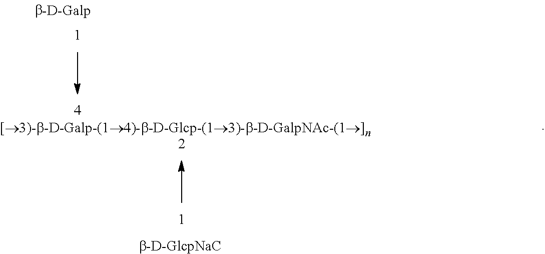

wherein each n is independently an integer of 1 to 40, preferably 5 to 30, preferably 7 to 25.

In certain embodiments, the PglB in prokaryotic host cell for preparing a bioconjugate of an E. coli O21 comprises the amino acid mutation N311V relative to wild-type PglB having the amino acid sequence of SEQ ID NO: 4. The invention also comprises a method of preparing a bioconjugate of an E. coli O21 antigen polysaccharide covalently linked to a carrier protein, the method comprising culturing the recombinant prokaryotic host cell as described herein to produce the bioconjugate.

### BRIEF DESCRIPTION OF THE DRAWINGS

FIG. 1: IgG responses induced by O153-EPA bioconjugate. Sprague Dawley rats were immunized intramuscularly 3 times with formulation buffer or O153-EPA conjugate at 3 different doses (0.04 μg, 0.40 μg or 4.00 μg O153-EPA). Serum Ab levels were measured by ELISA at day 0, 14 and 42 post-immunization. Individual titers (log 10 EC50 titer) and GMT±95% CI are shown. The dotted line indicates the threshold above which the dilution curves of the samples have a 4PL fitting.

FIG. 2: IgG responses induced by O21-EPA bioconjugate. Sprague Dawley rats were immunized intramuscularly 3 times with formulation buffer or O21-EPA bioconjugate at 3 different doses (0.04 μg, 0.40 μg or 4.00 μg O21-EPA). Serum Ab levels were measured by ELISA at day 0, 14 and 42 post-immunization. Individual titers (log 10 EC50 titer) and GMT±95% CI are shown. The dotted line indicates the threshold above which the dilution curves of the samples have a 4PL fitting.

FIG. 3: Functionality of antibodies induced by O153-EPA bioconjugate.

Sprague Dawley rats were immunized 3 times with formulation buffer or 4.00 μg/dose O153-EPA bioconjugate intramuscularly. Killing of O153 bacteria mediated by antibodies at day 42 post-immunization was measured by OPKA. Individual opsonization index (OI) values and GMT±95% CI are shown. ***p<0.001, Wilcoxon rank sum test using Bonferroni correction for multiple comparisons in accordance with the analysis of ELISA results.

FIG. 4: Functionality of antibodies induced by O21-EPA bioconjugate.

Sprague Dawley rats were immunized 3 times with formulation buffer or 4.00 μg/dose O21-EPA bioconjugate intramuscularly. Killing of O21 bacteria mediated by antibodies at day 42 post-immunization was measured by OPKA. Individual opsonization index (OI) values and GMT±95% CI are shown. ***p<0.001, Wilcoxon rank sum test using Bonferroni correction for multiple comparisons in accordance with the analysis of ELISA results.

FIGS. 5A-5L: Serum IgG responses induced by the ExPEC12V vaccine. Sprague Dawley rats were immunized intramuscularly 3 times with 4/8 μg PS/dose of ExPEC12V (4 μg for all O-antigens except O25 which is 8 μg), 0.4/0.8 μg PS/dose (0.4 μg for all O-antigens except O25 which is 0.8 μg) of ExPEC12V or formulation buffer. Levels of serum Abs specific for O1A (FIG. 5A), O2 (FIG. 5B), O4 (FIG. 5C), O6A (FIG. 5D), O8 (FIG. 5E), O15 (FIG. 5F), O16 (FIG. 5G), O18 (FIG. 5H), O21 (FIG. 5I), O25B (FIG. 5J), O75 (FIG. 5K) or O153 (FIG. 5L) were measured by ELISA at day 0, 28 and 42 postimmunization.

Individual titers (EC50 titer) and GMT±95% CI are shown.

### DESCRIPTION OF THE INVENTION

### Definitions

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning commonly understood to one of ordinary skill in the art to which this invention pertains. Otherwise, certain terms cited herein have the meanings as set in the specification. It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series.

The term “about,” when used in conjunction with a number, refers to any number within ±10%, e.g. ±5%, or ±1%, of the referenced number.

Those skilled in the art will recognize or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising,” “containing,” “including,” and “having,” whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the terms “O polysaccharide,” “O-antigen”, “O-antigen”, “O-antigen polysaccharide,” “O-polysaccharide antigen” and the abbreviation “OPS”, all refer to the O-antigen of Gram-negative bacteria, which is a component of the lipopolysaccharide (LPS) and is specific for each serotype or sero(sub)type of the Gram-negative bacteria. The O-antigen usually contains repeating units (RUs) of two to seven sugar residues. As used herein, the RU is set equal to the biological repeat unit (BRU). The BRU describes the RU of an O-antigen as it is synthesized in vivo. Different serotypes of E. coli express different O-antigens. In E. coli, the gene products involved in O-antigen biogenesis are encoded by the rfb gene cluster. Whenever referring to an O-antigen polysaccharide herein, the O-antigen polysaccharide of the respective E. coli serotype and any existing subserotypes thereof are meant unless indicated otherwise, e.g. when referring to O1 antigen polysaccharide, this can be O-antigen polysaccharide of E. coli subserotypes O1A, O1A1, O1B, or O1C, while O25 antigen polysaccharide can mean E. coli O25A or O25B antigen polysaccharide, etc. Many E. coli serotypes and subserotypes as well as the corresponding structure of a RU (moiety structure, or O-unit) of the O-antigen polysaccharides thereof are provided in Table 1 of WO 2020/039359, incorporated by reference herein.

As used herein, “rfb cluster” and “rfb gene cluster” refer to a gene cluster that encodes enzymatic machinery capable of synthesizing an O-antigen backbone structure. The term rfb cluster can apply to any O-antigen biosynthetic cluster, and preferably refers to a gene cluster from the genus Escherichia, particularly E. coli.

As used herein, the term “O1A” refers to the O1A antigen of E. coli (a subserotype of E. coli serotype O1). The term “O2” refers to the O2 antigen of E. coli (E. coli serotype O2). The term “O4” refers to the O4 antigen of E. coli (E. coli serotype O4). The term “O6A” refers to the O6A antigen of E. coli (a subserotype of E. coli serotype O6). The term “O8” refers to the O8 antigen of E. coli (E. coli serotype O8). The term “O15” refers to the O15 antigen of E. coli (E. coli serotype O15). The term “O16” refers to the O16 antigen of E. coli (E. coli serotype O16). The term “O18A” refers to the O18A antigen of E. coli (a subserotype of E. coli serotype O18). The term “O25B” refers to the O25B antigen from E. coli (a subserotype of E. coli serotype O25). The term “O75” refers to the O75 antigen of E. coli (E. coli serotype O75). The term “O153” refers to the O153 antigen of E. coli (E. coli serotype O153). The term “O21” refers to the O21 antigen of E. coli (E. coli serotype O21). As used herein the term “O4A” refer to O4 O-antigen of E. coli (E. coli serotype O4) with a glucose side-branch (this term was previously described as “O4 Glc+” in WO2020191082 and WO2022/058945).

The structures of several E. coli O-antigen polysaccharides referred to throughout this application are shown below in Table 1. A single repeating unit for each E. coli O-antigen polysaccharide is shown.

TABLE 1
Structures of E. coli O-antigen Polysaccharides

| E. coli O- | |
|---|---|
| antigen | |
| Poly- | |
| saccharide | Structure of Repeating Unit¹ |
| O1A antigen polysaccharide (O1A) | 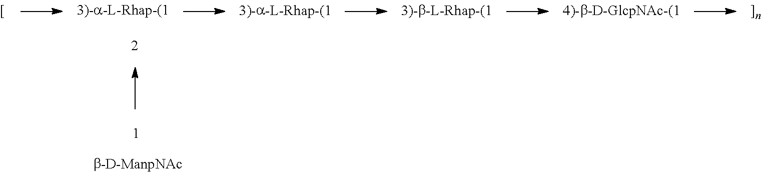 |
| O2 antigen polysaccharide (O2) | 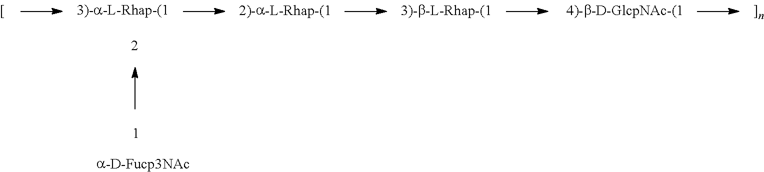 |
| Non- | [→2)-α-L-Rhap-(1→6)-α-D-Glcp-(1→3)-α-L-FucpNAc-(1→3)-β-D-GlcpNAc-(1→]ₙ |
| glucosylated | |
| O4 antigen | |
| polysaccharide | |
| (O4-Glc-) | |
| Glucosylated O4 antigen polysaccharide (O4-Glc+; O4A) | 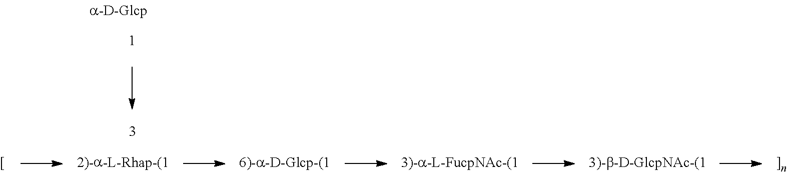 |
| O6A antigen polysaccharide (O6A) | 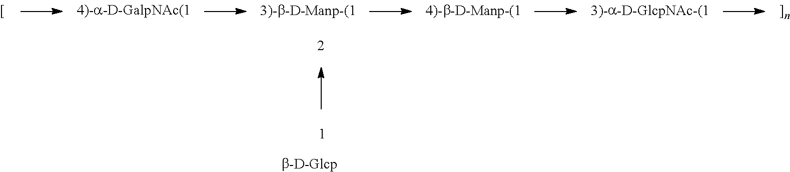 |
| O8 antigen | α-D-Manp3Me-(1→[3)-β-D-Manp-(1→2)-α-D-Manp-(1→2)-α-D-Manp-(1→]ₙ |
| polysaccharide | |
| (O8) | |
| O15 antigen | [→2)-β-D-Galp-(1→3)-α-L-FucpNAc-(1→3)-β-D-GlcpNAc-(1→]ₙ |
| polysaccharide | |
| (O15) | |
| O16 antigen polysaccharide (O16) | 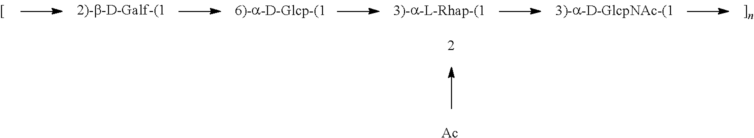 |
| O18A antigen polysaccharide (O18A) | 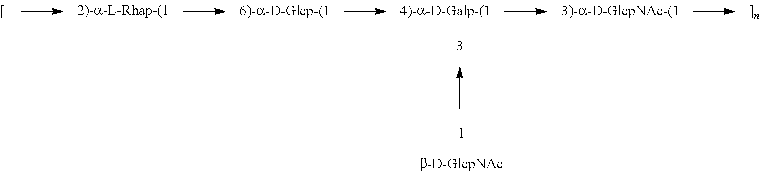 |
| O25B antigen polysaccharide (O25B) | 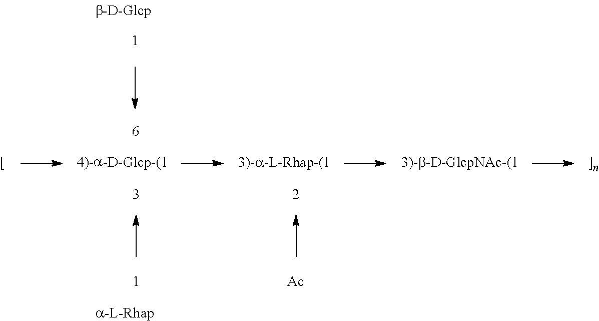 |
| 075 antigen polysaccharide (O75) | 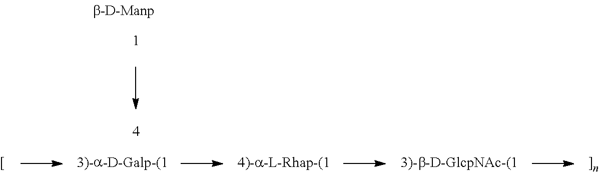 |
| O153 antigen | [→2)-β-D-Ribf-(1→4)-β-D-Galp-(1→4)-α-D-GlcpNAc-(1→4)-β-b-Galp-(1→3)-α-D-GlcpNAc-(1→]ₙ |
| polysaccharide | |
| (O153) | |
| O21 antigen polysaccharide (O21) | 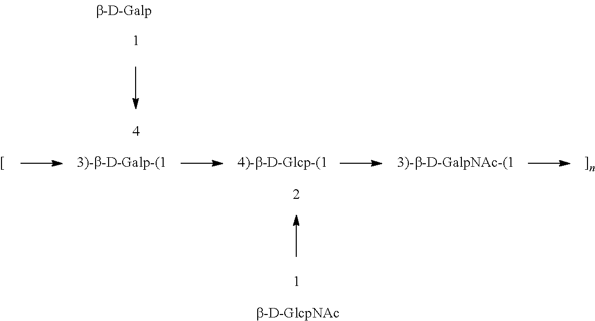 |

¹Each n is independently an integer of 1 to 100, such as 1-50, 1-40, 1-30, 1-20, and 1-10, 3-50, 3-40, 5-30, e.g. at least 5, such as 5-40, e.g. 7-30, e.g. 7 to 25, e.g. 10 to 20, but in some instances can be 1-2.

All monosaccharides described herein have their common meaning known in the art. Monosaccharides can have the D or L configuration. If D or L is not specified, the sugar is understood to have the D configuration. Monosaccharides are typically referred to by abbreviations commonly known and used in the art. For example, Glc refers to glucose; D-Glc refers to D-glucose; and L-Glc refers to L-glucose. Other common abbreviations for monosaccharides include: Rha, rhamnose; GlcNAc, N-acetylglucosamine; GalNAc, N-acetylgalactosamine; Fuc, fucose; Man, mannose; Man3Me, 3-O-methyl-mannose; Gal, galactose; FucNAc, N-acetylfucosamine; and Rib, ribose. The suffix “f” refers to furanose and the suffix “p” refers to pyranose.

The terms “RU,” “repeat unit,” and “repeating unit” as used with respect to an O-antigen refer to the biological repeat unit (BRU) of an O-antigen as it is synthesized in vivo by cellular machinery (e.g., glycosyltransferases). The number of RUs of an O-antigen may vary per serotype, and in embodiments of the invention typically varies from about 1-100 RUs, preferably about 1 to 50 RUs, such as 1-50 RUs, 1-40 RUs, 1-30 RUs, 1-20 RUs, and 1-10 RUs, and more preferably at least 3 RUs, at least 4 RUs, at least 5 RUs, such as 3-50 RUs, preferably 5-40 RUs, preferably 5-30 RUs, e.g. 7-25 RUs, e.g. 10-20 RUs. However, in some instances, the number of RUs of an O-antigen can be 1-2. The structure of each O-antigen that is specifically described herein is shown containing one RU with the variable “n” designating the number of RUs. In each O-antigen polysaccharide in a bioconjugate of the invention, n is independently an integer of 1-100, such as 1-50, 1-40, 1-30, 1-20, 1-10, preferably at least 3, more preferably at least 5, such as 3-50, preferably 5-40 (e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40), more preferably 5-30, but in some instances can be 1-2. In some embodiments n is independently an integer of about 7-25, e.g. about 10-20. The values may vary between individual O-antigen polysaccharides in a composition, and are provided here as average values, i.e. if a bioconjugate is described herein as having an n that is independently an integer of 5-40, the composition contains a majority of O-antigen polysaccharides with 5-40 repeat units, but may also contain some O-antigen polysaccharides that have less than 5 repeat units or more than 40 repeat units.

As used herein, the terms “conjugate” and “glycoconjugate” refer to a sugar or saccharide antigen (e.g., oligo- and polysaccharide)-protein conjugate linked to another chemical species, including but not limited to proteins, peptides, lipids, etc. Glycoconjugates can be prepared chemically, e.g., by chemical (synthetic) linkage of the protein and sugar or saccharide antigen. The term glycoconjugate also includes bioconjugates.

As used herein, the term “effective amount” in the context of administering an O-antigen to a subject in methods according to embodiments of the invention refers to the amount of the O-antigen that is sufficient to induce a desired immune effect or immune response in the subject. In certain embodiments, an “effective amount” refers to the amount of an O-antigen which is sufficient to produce immunity in a subject to achieve one or more of the following effects in the subject: (i) prevent the development or onset of an ExPEC infection, preferably an invasive ExPEC disease, or symptom associated therewith; (ii) prevent the recurrence of an ExPEC infection, preferably an invasive ExPEC disease, or symptom associated therewith; (iii) prevent, reduce or ameliorate the severity of an ExPEC infection, preferably an invasive ExPEC disease, or symptom associated therewith; (iv) reduce the duration of an ExPEC infection, preferably an invasive ExPEC disease, or symptom associated therewith; (v) prevent the progression of an ExPEC infection, preferably an invasive ExPEC disease, or symptom associated therewith; (vi) cause regression of an ExPEC infection or symptom associated therewith; (vii) prevent or reduce organ failure associated with an ExPEC infection; (viii) reduce the chance or frequency of hospitalization of a subject having an ExPEC infection; (ix) reduce hospitalization length of a subject having an ExPEC infection; (x) increase the survival of a subject with an ExPEC infection, preferably an invasive ExPEC disease; (xi) eliminate an ExPEC infection, preferably an invasive ExPEC disease; (xii) inhibit or reduce ExPEC replication; and/or (xiii) enhance or improve the prophylactic or therapeutic effect(s) of another therapy.

An “effective amount” can vary depending upon a variety of factors, such as the physical condition of the subject, age, weight, health, etc.; route of administration, such as oral or parenteral; the composition administered, such as the target O-antigen, the other co-administered O-antigens, adjuvant, etc.; and the particular disease for which immunity is desired. When the O-antigen is covalently bound to a protein carrier, the effective amount for the O-antigen is calculated based on only the O-antigen polysaccharide moiety in the conjugate. All concentrations, amounts, and ratios of conjugates, including bioconjugates, as described herein, are also calculated based only on the weight of the O-antigen polysaccharide moieties in the conjugates, regardless of the concentration, amount, or ratio of any conjugated carrier proteins, unless indicated otherwise. For example, administration of 16 μg of a particular bioconjugate means that the administered bioconjugate comprises 16 μg of the particular O-antigen polysaccharide, and the amount of the conjugated carrier protein is not included in this number. For another example, if a composition is said to comprise conjugates of O-antigen polysaccharides from serotypes A and B in a ratio of 2:1, it indicates that there is two times the concentration or amount of the conjugated O-antigen polysaccharide A than that of the conjugated O-antigen polysaccharide B based on the weight of the conjugated O-antigen polysaccharides, disregarding the weight of the conjugated carrier proteins, in the composition.

The term “Invasive Extraintestinal pathogenic Escherichia coli (ExPEC) disease (IED)” as used herein is an acute illness consistent with systemic bacterial infection, which is microbiologically confirmed either by the isolation and identification of E. coli from blood or other normally sterile body sites, or by the isolation and identification of E. coli from urine in a patient with presence of signs and symptoms of invasive disease (systemic inflammatory response syndrome (SIRS), sepsis or septic shock) and no other identifiable source of infection. In certain embodiments, IED is an acute illness consistent with systemic bacterial infection, which is microbiologically confirmed either by (i) the isolation and identification of E. coli from blood or other normally sterile body sites, or by (ii) the isolation and identification of E. coli from urine in a patient with life threatening organ dysfunction due to dysregulated host response to infection originating from the urinary tract and/or male genital organs and no other identifiable source of infection.

IED may include, but is not necessarily limited to, urinary tract infection (UTI), a surgical-site infection, an abdominal or pelvic infection, pneumonia, osteomyelitis, cellulitis, sepsis, bacteremia, a wound infection, pyelonephritis, prostate biopsy-related infection (such as transrectal ultrasound-guided prostate needle biopsy [TRUS-PNB] related infection), urosepsis, meningitis, peritonitis, cholangitis, soft-tissue infections, pyomyositis, septic arthritis, endophthalmitis, suppurative thyroiditis, sinusitis, endocarditis, neutropenic fever, and prostatitis (including but not limited to acute bacterial prostatitis).

In certain preferred embodiments, IED comprises sepsis. In certain preferred embodiments, IED comprises bacteremia. The invention in certain embodiments provides a composition according to the invention for preventing sepsis caused by E. coli. The invention in certain embodiments provides a composition according to the invention for preventing bacteremia caused by E. coli.

The term “IED event meeting criteria for sepsis” indicates an IED case including evidence of life-threatening organ dysfunction due to dysregulated host response to infection. A case of IED is meeting criteria for sepsis if there is an acute change in total Sequential Organ Failure Assessment (SOFA) score of 2 points or greater from baseline and deemed secondary to the IED. The invention in certain embodiments provides a composition according to the invention for preventing IED meeting the criteria for sepsis. The term “urosepsis” as used herein is sepsis caused by an infection originating from the urogenital tract and/or male genital organs.

The term “bacteremic IED” is an IED case which includes isolation and identification of E. coli from blood. The invention in certain embodiments provides a composition according to the invention for preventing bacteremic IED.

As used herein, an “immunological response” or “immune response” to an antigen or composition refers to the development in a subject of a humoral and/or a cellular immune response to the antigen or an antigen present in the composition.

As used herein, a “composition” comprising more than one E. coli antigen polysaccharide can be a single pharmaceutical composition that comprises the more than one E. coli antigen polysaccharide in the same pharmaceutical composition, or a combination of more than one pharmaceutical composition that comprises the more than one E. coli antigen polysaccharide in separate pharmaceutical compositions. In preferred embodiments, a composition is a single pharmaceutical composition. In a method of inducing an immune response to E. coli, a “composition” comprising more than one E. coli antigen polysaccharide can be administered to a subject in need thereof together in a single pharmaceutical composition that comprises the more than one E. coli antigen polysaccharide or can be administered to the subject in combination in separate pharmaceutical compositions. In preferred embodiments, a single pharmaceutical composition is administered to the subject.

As used herein, the terms “in combination,” or “a combination of” in the context of the administration of two or more O-antigens or compositions to a subject, does not restrict the order in which O-antigens or compositions are administered to a subject. For example, a first composition can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second composition to a subject. Preferably the two or more O-antigens are administered to a subject essentially simultaneously, e.g. within five minutes of each other, and more preferably the two or more O-antigens are administered simultaneously via administration of at least two compositions at the same time, most preferably via administration of a single composition that comprises the two or more O-antigens.

As used herein, “subject” means any animal, preferably a mammal, most preferably a human, to who will be or has been vaccinated by a method or composition according to an embodiment of the invention. The term “mammal” as used herein, encompasses any mammal. Examples of mammals include, but are not limited to, cows, horses, sheep, pigs, cats, dogs, mice, rats, rabbits, guinea pigs, monkeys, humans, etc., most preferably a human. The terms “subject” and “patient” may be used herein interchangeably.

The term “percent (%) sequence identity” or “% identity” describes the number of matches (“hits”) of identical amino acids of two or more aligned amino acid sequences as compared to the number of amino acid residues making up the overall length of the amino acid sequences. In other terms, using an alignment, for two or more sequences the percentage of amino acid residues that are the same (e.g. 90%, 95%, 97% or 98% identity) may be determined, when the sequences are compared and aligned for maximum correspondence as measured using a sequence comparison algorithm as known in the art, or when manually aligned and visually inspected. The sequences which are compared to determine sequence identity may thus differ by substitution(s), addition(s) or deletion(s) of amino acids. Suitable programs for aligning protein sequences are known to the skilled person. The percentage sequence identity of protein sequences can, for example, be determined with programs such as CLUSTALW, Clustal Omega, FASTA or BLAST, e.g using the NCBI BLAST algorithm (Altschul S F, et al (1997), Nucleic Acids Res. 25:3389-3402).

For example, for amino acid sequences, sequence identity and/or similarity can be determined by using standard techniques known in the art, including, but not limited to, the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al, 1984, Nucl. Acid Res. 12:387-395, preferably using the default settings, or by inspection. In certain embodiments, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc.

Another example of a useful algorithm is the BLAST algorithm, described in: Altschul et al, 1990, J. Mol. Biol. 215:403-410; Altschul et al, 1997, Nucleic Acids Res. 25:3389-3402; and Karin et al, 1993, Proc. Natl. Acad. Sci. U.S.A. 90:5873-5787. A particularly useful BLAST program is the WU-BLAST-2 program which was obtained from Altschul et al, 1996, Methods in Enzymology 266:460-480. WU-BLAST-2 uses several search parameters, most of which are set to the default values.

An additional useful algorithm is gapped BLAST as reported by Altschul et al, 1993, Nucl. Acids Res. 25:3389-3402.

It is shown herein that vaccination or immunisation with compositions comprising E. coli O1, O2, O4, O6, O8, O15, O16, O18, O25, and O75 antigen polysaccharides, and further comprising O153, or O21, or O153 and O21 antigen polysaccharides, each independently covalently linked to a carrier protein, induces a solid and robust immune response against all of O1, O2, O4, O6, O8, O15, O16, O18, O25, and O75, as well as to O153 and/or O21, which was unpredictable prior to the instant invention.

Accordingly, in a first aspect, the invention provides for a composition comprising E. coli O1, O2, O4, O6, O8, O15, O16, O18, O25, O75 and O153 antigen polysaccharides, wherein each of the antigen polysaccharides is independently covalently linked to a carrier protein.

In certain embodiments, the concentration of O25 and/or O75 antigen polysaccharides is independently increased relative to the concentration of each of O1, O2, O4, O6, O8, O15, O16, O18, and O153 antigen polysaccharides.

In certain embodiments, the weight ratio of concentrations of O25 antigen polysaccharide independently to each of O1, O2, O4, O6, O8, O15, O16, O18, O75 and O153 antigen polysaccharides is about 1.5:1 to about 2.5:1.

In certain embodiments, the weight ratio of concentrations of O25 antigen polysaccharide independently to each of O1, O2, O4, O6, O8, O15, O16, O18, O75 and O153 antigen polysaccharides is about 2:1.

In certain embodiments, the weight ratio of concentrations of O25 and O75 antigen polysaccharide independently to each of O1, O2, O4, O6, O8, O15, O16, O18 and O153 antigen polysaccharides is about 1.5:1 to about 2.5:1.

In certain embodiments, the weight ratio of concentrations of O25 and O75 antigen polysaccharide independently to each of O1, O2, O4, O6, O8, O15, O16, O18 and O153 antigen polysaccharides is about 2:1.

In certain embodiments, the weight ratio of concentrations of the E. coli antigen polysaccharides O1:O2:O4:O6:O8:O15:O16:O18:O153:O25:O75 is 1:1:1:1:1:1:1:1:1:2:2.

In a further aspect, the invention provides for a composition comprising E. coli O1, O2, O4, O6, O8, O15, O16, O18, O25, O75 and O21 antigen polysaccharides, wherein each of the antigen polysaccharides is independently covalently linked to a carrier protein.

In certain embodiments, the concentration of O25 and/or O75 antigen polysaccharides is independently increased relative to the concentration of each of O1, O2, O4, O6, O8, O15, O16, O18, and O21 antigen polysaccharides.

In certain embodiments, the weight ratio of concentrations of O25 antigen polysaccharide independently to each of O1, O2, O4, O6, O8, O15, O16, O18, O75 and O21 antigen polysaccharides is about 1.5:1 to about 2.5:1.

In certain embodiments, the weight ratio of concentrations of O25 antigen polysaccharide independently to each of O1, O2, O4, O6, O8, O15, O16, O18, O75 and O21 antigen polysaccharides is about 2:1.

In certain embodiments, the weight ratio of concentrations of O25 and O75 antigen polysaccharide independently to each of O1, O2, O4, O6, O8, O15, O16, O18 and O21 antigen polysaccharides is about 1.5:1 to about 2.5:1.

In certain embodiments, the weight ratio of concentrations of O25 and O75 antigen polysaccharide independently to each of O1, O2, O4, O6, O8, O15, O16, O18 and O21 antigen polysaccharides is about 2:1.

In certain embodiments, the weight ratio of concentrations of the E. coli antigen polysaccharides O1:O2:O4:O6:O8:O15:O16:O18:O21:O25:O75 is 1:1:1:1:1:1:1:1:1:2:2.

In yet a further aspect, the invention provides for a composition comprising E. coli O1, O2, O4, O6, O8, O15, O16, O18, O25, O75, O153 and O21 antigen polysaccharides, wherein each of the antigen polysaccharides is independently covalently linked to a carrier protein.

In certain embodiments, the concentration of O25 and/or O75 antigen polysaccharides is independently increased relative to the concentration of each of O1, O2, O4, O6, O8, O15, O16, O18, O153 and O21 antigen polysaccharides.

In certain embodiments, the weight ratio of concentrations of O25 antigen polysaccharide independently to each of O1, O2, O4, O6, O8, O15, O16, O18, O75, O153 and O21 antigen polysaccharides is about 1.5:1 to about 2.5:1.

In certain embodiments, the weight ratio of concentrations of O25 antigen polysaccharide independently to each of O1, O2, O4, O6, O8, O15, O16, O18, O75, O153 and O21 antigen polysaccharides is about 2:1.

In certain embodiments, the weight ratio of concentrations of O25 and O75 antigen polysaccharide independently to each of O1, O2, O4, O6, O8, O15, O16, O18, O153 and O21 antigen polysaccharides is about 1.5:1 to about 2.5:1.

In certain embodiments, the weight ratio of concentrations of O25 and O75 antigen polysaccharide independently to each of O1, O2, O4, O6, O8, O15, O16, O18, O153 and O21 antigen polysaccharides is about 2:1.

In certain embodiments, the weight ratio of concentrations of the E. coli antigen polysaccharides O1:O2:O4:O6:O8:O15:O16:O18:O21:O153:O25:O75 is 1:1:1:1:1:1:1:1:1:1:2:2.

In certain embodiments according to the invention, the O1 antigen is O1A, the O4 is glucosylated (O4A), the O6 antigen is O6A, the O18 antigen is O18A, and the O25 antigen is O25B. In certain embodiments, the O1A, O2, glucosylated O4, O6A, O15, O16, O18A, O25B, O75, O21 and O153 antigen polysaccharides comprise the structures of Formulas (O1A), (O2), (O4A), (O6A), (O15), (O16), (O18A), (O25B), (O75), (O21) and (O153) respectively, as shown in Table 1, wherein each n is independently an integer of 1 to 100, preferably of 3 to 50, for example 5 to 40, preferably of 5 to 30, for example 7 to 25, for example 10 to 20.

In one embodiment, an O1 antigen polysaccharide is used in a composition provided herein. In a specific embodiment, the O1 antigen polysaccharide comprises the structure of formula (O1A) as shown in Table 1, wherein n is an integer of 1-100, preferably 3-50, e.g. 5-40, preferably 5-30, e.g. 7 to 25, e.g. 10 to 20. Preferably, the O1 antigen polysaccharide is part of a chemical conjugate or bioconjugate and is covalently linked to a carrier protein, e.g., EPA or CRM197. In a preferred embodiment, the O1 antigen polysaccharide is covalently linked to an Asn residue in an EPA carrier protein, preferably obtained via bioconjugation.

In one embodiment, an O2 antigen polysaccharide is used in a composition provided herein. In a specific embodiment, the O2 antigen polysaccharide comprises the structure of formula (O2) as shown in Table 1, wherein n is an integer of 1-100, preferably 3-50, e.g. 5-40, preferably 5-30, e.g. 7 to 25, e.g. 10 to 20. Preferably, the O2 antigen polysaccharide is part of a chemical conjugate or bioconjugate and is covalently linked to a carrier protein, e.g., EPA or CRM197. In a preferred embodiment, the O2 antigen polysaccharide is covalently linked to an Asn residue in an EPA carrier protein, preferably obtained via bioconjugation.

In one embodiment, an O4 antigen polysaccharide is used in a composition provided herein. In a specific embodiment, the O4 antigen polysaccharide is a glucosylated O4 antigen polysaccharide, and in a specific embodiment comprises the structure of formula (O4A) as shown in Table 1, wherein n is an integer of 1-100, preferably 3-50, e.g. 5-40, preferably 5-30, e.g. 7 to 25, e.g. 10 to 20. Preferably, the O4 antigen polysaccharide is part of a chemical conjugate or bioconjugate and is covalently linked to a carrier protein, e.g., EPA or CRM₁₉₇. In a preferred embodiment, the O4 antigen polysaccharide is covalently linked to an Asn residue in an EPA carrier protein, preferably obtained via bioconjugation.

In one embodiment, an O6 antigen polysaccharide is used in a composition provided herein. In a specific embodiment, the O6 antigen polysaccharide comprises the structure of formula (O6A) as shown in Table 1, wherein n is an integer of 1-100, preferably 3-50, e.g. 5-40, preferably 5-30, e.g. 7 to 25, e.g. 10 to 20. Preferably, the O6 antigen polysaccharide is part of a chemical conjugate or bioconjugate and is covalently linked to a carrier protein, e.g., EPA or CRM₁₉₇. In a preferred embodiment, the O6 antigen polysaccharide is covalently linked to an Asn residue in an EPA carrier protein, preferably obtained via bioconjugation.

In one embodiment, an O8 antigen polysaccharide is used in a composition provided herein. In a specific embodiment, the O8 antigen polysaccharide comprises the structure of formula (O8) as shown in Table 1, wherein n is an integer of 1-100, preferably 3-50, e.g. 5-40, preferably 5-30, e.g. 7 to 25, e.g. 10 to 20. Preferably, the O8 antigen polysaccharide is part of a chemical conjugate or bioconjugate and is covalently linked to a carrier protein, e.g., EPA or CRM₁₉₇. In a preferred embodiment, the O8 antigen polysaccharide is covalently linked to an Asn residue in an EPA carrier protein, preferably obtained via bioconjugation.

In one embodiment, an O15 antigen polysaccharide is used in a composition provided herein. In a specific embodiment, the O15 antigen polysaccharide comprises the structure of formula (O15) as shown in Table 1, wherein n is an integer of 1-100, preferably 3-50, e.g. 5-40, preferably 5-30, e.g. 7 to 25, e.g. 10 to 20. Preferably, the O15 antigen polysaccharide is part of a chemical conjugate or bioconjugate and is covalently linked to a carrier protein, e.g., EPA or CRM₁₉₇. In a preferred embodiment, the O15 antigen polysaccharide is covalently linked to an Asn residue in an EPA carrier protein, preferably obtained via bioconjugation.

In one embodiment, an O16 antigen polysaccharide is used in a composition provided herein. In a specific embodiment, the O16 antigen polysaccharide comprises the structure of formula (O16) as shown in Table 1, wherein n is an integer of 1-100, preferably 3-50, e.g. 5-40, preferably 5-30, e.g. 7 to 25, e.g. 10 to 20. Preferably, the O16 antigen polysaccharide is part of a chemical conjugate or bioconjugate and is covalently linked to a carrier protein, e.g., EPA or CRM₁₉₇. In a preferred embodiment, the O16 antigen polysaccharide is covalently linked to an Asn residue in an EPA carrier protein, preferably obtained via bioconjugation.

In one embodiment, an O18 antigen polysaccharide is used in a composition provided herein. In a specific embodiment, the O18 antigen polysaccharide comprises the structure of formula (O18A) as shown in Table 1, wherein n is an integer of 1-100, preferably 3-50, e.g. 5-40, preferably 5-30, e.g. 7 to 25, e.g. 10 to 20. Preferably, the O18 antigen polysaccharide is part of a chemical conjugate or bioconjugate and is covalently linked to a carrier protein, e.g., EPA or CRM₁₉₇. In a preferred embodiment, the O18 antigen polysaccharide is covalently linked to an Asn residue in an EPA carrier protein, preferably obtained via bioconjugation.

In one embodiment, an O25 antigen polysaccharide is used in a composition provided herein. In a specific embodiment, the O25 antigen polysaccharide comprises an O25B antigen polysaccharide, and in a specific embodiment comprises the structure of formula (O25B) as shown in Table 1, wherein n is an integer of 1-100, preferably 3-50, e.g. 5-40, preferably 5-30, e.g. 7 to 25, e.g. 10 to 20. Preferably, the O25 antigen polysaccharide is part of a chemical conjugate or bioconjugate and is covalently linked to a carrier protein, e.g., EPA or CRM₁₉₇. In a preferred embodiment, the O25 antigen polysaccharide is covalently linked to an Asn residue in an EPA carrier protein, preferably obtained via bioconjugation.

In one embodiment, an O75 antigen polysaccharide is used in a composition provided herein. In a specific embodiment, the O75 antigen polysaccharide comprises an O75 antigen polysaccharide, and in a specific embodiment comprises the structure of formula (O75) as shown in Table 1, wherein n is an integer of 1-100, preferably 3-50, e.g. 5-40, preferably 5-30, e.g. 7 to 25, e.g. 10 to 20. Preferably, the O75 antigen polysaccharide is part of a chemical conjugate or bioconjugate and is covalently linked to a carrier protein, e.g., EPA or CRM₁₉₇. In a preferred embodiment, the O75 antigen polysaccharide is covalently linked to an Asn residue in an EPA carrier protein, preferably obtained via bioconjugation.

In one embodiment, an O153 antigen polysaccharide is used in a composition provided herein. In a specific embodiment, the O153 antigen polysaccharide comprises an O153 antigen polysaccharide, and in a specific embodiment comprises the structure of formula (O153) as shown in Table 1, wherein n is an integer of 1-100, preferably 3-50, e.g. 5-40, preferably 5-30, e.g. 7 to 25, e.g. 10 to 20. Preferably, the O153 antigen polysaccharide is part of a chemical conjugate or bioconjugate and is covalently linked to a carrier protein, e.g., EPA or CRM₁₉₇. In a preferred embodiment, the O153 antigen polysaccharide is covalently linked to an Asn residue in an EPA carrier protein, preferably obtained via bioconjugation.

In one embodiment, an O21 antigen polysaccharide is used in a composition provided herein. In a specific embodiment, the O21 antigen polysaccharide comprises an O21 antigen polysaccharide, and in a specific embodiment comprises the structure of formula (O21) as shown in Table 1, wherein n is an integer of 1-100, preferably 3-50, e.g. 5-40, preferably 5-30, e.g. 7 to 25, e.g. 10 to 20. Preferably, the O21 antigen polysaccharide is part of a chemical conjugate or bioconjugate and is covalently linked to a carrier protein, e.g., EPA or CRM₁₉₇. In a preferred embodiment, the O21 antigen polysaccharide is covalently linked to an Asn residue in an EPA carrier protein, preferably obtained via bioconjugation.

In certain embodiments, composition as described herein further comprises at least one additional E. coli O-antigen polysaccharide covalently linked to a carrier protein.

In certain embodiments, the composition as described herein consists essentially of E. coli O antigen polysaccharides O1, O2, O4, O6, O8, O15, O16, O18, O25, O75 and O153 covalently linked to a carrier protein. In such a composition, other components may optionally be present, but the composition does not comprise further E. coli O-antigen polysaccharides.

In certain embodiments, the composition as described herein consists essentially of E. coli O antigen polysaccharides O1, O2, O4, O6, O8, O15, O16, O18, O25, O75 and O21 covalently linked to a carrier protein. In such a composition, other components may optionally be present, but the composition does not comprise further E. coli O-antigen polysaccharides.

In certain embodiments, the composition as described herein consists essentially of E. coli O antigen polysaccharides O1, O2, O4, O6, O8, O15, O16, O18, O25, O75, O153 and O21 covalently linked to a carrier protein. In such a composition, other components may optionally be present, but the composition does not comprise further E. coli O-antigen polysaccharides.

In certain embodiments, each carrier protein is independently selected from the group consisting of detoxified Exotoxin A of P. aeruginosa (EPA), E. coli flagellin (FliC), CRM₁₉₇, maltose binding protein (MBP), Diphtheria toxoid, Tetanus toxoid, detoxified hemolysin A of S. aureus, clumping factor A, clumping factor B, E. coli heat labile enterotoxin, detoxified variants of E. coli heat labile enterotoxin, Cholera toxin B subunit (CTB), cholera toxin, detoxified variants of cholera toxin, E. coli Sat protein, the passenger domain of E. coli Sat protein, Streptococcus pneumoniae Pneumolysin, Keyhole limpet hemocyanin (KLH), P. aeruginosa PcrV, outer membrane protein of Neisseria meningitidis (OMPC), and protein D from non-typeable Haemophilus influenzae.

In a particular embodiment, the carrier protein is detoxified exotoxin A of Pseudomonas aeruginosa (EPA) or CRM₁₉₇. The amino acid sequence of a CRM₁₉₇ protein is shown in SEQ ID NO: 5.

For preparation of bioconjugates, the carrier protein is preferably encoded in the host cell, e.g. on a plasmid.

In particular embodiments, a carrier protein is a detoxified Exotoxin A of P. aeruginosa. For EPA, various detoxified protein variants have been described in literature and could be used as carrier proteins. In certain embodiments, the EPA carrier protein comprises 1-20 glycosylation sites, preferably 1-10 glycosylation sites, preferably 2-4, and more preferably 4 glycosylation sites each comprising a glycosylation consensus sequence having the amino acid sequence Asn-X-Ser(Thr), wherein X can be any amino acid except Pro, and more preferably the amino acid sequence of SEQ ID NO: 1.

In some embodiments, the EPA carrier protein comprises four glycosylation sites each comprising a glycosylation consensus sequence, for instance a glycosylation site comprising a glycosylation consensus sequence having SEQ ID NO: 1. As used herein, “EPA-4 carrier protein” and “EPA-4” refer to a detoxified Exotoxin A of P. aeruginosa carrier protein comprising four glycosylation sites each comprising a glycosylation consensus sequence having SEQ ID NO: 1. An exemplary preferred example of an EPA-4 carrier protein is EPA carrier protein comprising the amino acid sequence of SEQ ID NO: 2.

In certain embodiments, the E. coli antigen polysaccharides are covalently linked to the carrier protein by bioconjugation or by chemical conjugation. Chemical conjugation can, for example, include reductive amination chemistry (RAC), single-end conjugation, conjugation with a (2-((2-oxoethyl)thio)ethyl) carbamate (eTEC) spacer, cyanylation chemistry (CNBr, CDAP) with or without ADH spacer, thioether chemistry (maleimide/bromoacetyl linker based), or EDC-N-Hydroxy succinimide zero linker chemistry. Methods to make glycoconjugates of E. coli O-antigens conjugated to carrier proteins using chemical conjugation to carrier protein, and compositions comprising such glycoconjugates, have for instance also been described in WO 2020/039359 and WO2022/058645 which are incorporated herein by reference.

Preferably, the term “bioconjugate” refers to a conjugate between a protein (e.g., carrier protein) and an O-antigen, preferably an E. coli O-antigen (e.g., O1, O2, O4, O6, O8, O15, O16, O18, O25, O75, O21, O153 etc.) prepared in a host cell background, wherein host cell machinery links the antigen to the protein (e.g., N-links), preferably in vivo within the host cells (see e.g. examples herein and e.g. WO 03/074687, WO 2006/119987, WO 2015/124769, WO 2020/191082, WO 2020/191088, WO 2022/058945). Also conjugates that are prepared with lysates of such cells, i.e. in vitro but using the same glycosylation machinery (see e.g. WO 2017/117539, U.S. Pat. No. 10,829,795, WO 2020/146814), are considered bioconjugates. Because bioconjugates are prepared in or with host cells by host cell machinery, the antigen and protein are covalently linked via a glycosidic linkage or bond in a bioconjugate. Bioconjugates can be prepared in recombinant host cells (or lysates thereof) engineered to express the cellular machinery needed to synthesize the O-antigen and/or link the O-antigen to the target protein. Bioconjugates, as described herein, have advantageous properties over chemically prepared glycoconjugates where the glycans are purified from bacterial cell walls and subsequently chemically coupled to a carrier protein, e.g., bioconjugates require fewer chemicals in manufacture and are more consistent in terms of the final product generated, and contain less or no free (i.e. unbound to carrier protein) glycan. Purification of O-antigen free from lipid A and subsequent chemical conjugation to a carrier protein is a lengthy and laborious process. Additionally, the purification, lipid A detoxification and chemical conjugation processes can result in loss of epitopes, antigen heterogeneity and reduced immunogenicity of the conjugated polysaccharide. Synthesis of glycoconjugates by bioconjugation can overcome these limitations of classical purification and chemical conjugation. Thus, in typical embodiments, bioconjugates are preferred over chemically produced glycoconjugates. Preparation of bioconjugates for E. coli O1, O2, O6 and O25 antigens has been described in detail in WO 2015/124769 and WO 2017/035181. Preparation of bioconjugates for E. coli O4, in particular O4A, as well as bioconjugates for E. coli O1, O2, O6, O8, O15, O16, O18, O25, and O75 has been described in detail in WO2020/191082, preparation of bioconjugates for E. coli O18 has been described in detail in PCT/IB2022/053013, and preparation of conjugate compositions with E. coli O75 and other E. coli O-antigen conjugates has been described in detail in WO2022/058945.

Host cells suitable to prepare bioconjugates have for instance been described in WO2020/191082 and WO2022/058945.

Accordingly, in certain embodiments, the invention provides for a host cell (e.g. prokaryotic host cell) capable of producing a bioconjugate of an E. coli O153 antigen polysaccharide.

In certain embodiments, the invention provides for a host cell (e.g. prokaryotic host cell) capable of producing a bioconjugate of an E. coli O21 antigen polysaccharide.

The host cell provided herein is preferably modified to comprise (e.g., through genetic engineering) one or more of the nucleic acids encoding host cell machinery (e.g., glycosyltransferases) used to produce E. coli O153 antigen bioconjugates, or to produce E. coli O21 antigen bioconjugates, respectively. In certain embodiments the prokaryotic cells are gram negative bacteria. Exemplary prokaryotic host cells for use in production of the bioconjugates comprising the E. coli O153 or O21 antigen polysaccharide described herein include, but are not limited to, Escherichia species, Shigella species, Klebsiella species, Xhantomonas species, Salmonella species, Yersinia species, Lactococcus species, Lactobacillus species, Pseudomonas species, Corynebacterium species, Streptomyces species, Streptococcus species, Staphylococcus species, Bacillus species, and Clostridium species.

In certain embodiments, the host cell used to produce the O153 or O21 antigen polysaccharide bioconjugate is E. coli. In certain embodiments the host cell is a K-12 of E. coli (as a non-limiting example, E. coli strain W3110 is a K-12 strain), or a B strain of E. coli (as a non-limiting example, E. coli strain BL21 is a B strain), or any other well-defined strain of E. coli, e.g. laboratory strains or production strains, in contrast to primary wild-type isolates.

In certain embodiments, the host cell used to produce O153 antigen polysaccharides and bioconjugates described herein are engineered to comprise heterologous nucleic acids, e.g., heterologous nucleic acids comprising rfb gene clusters of the O153 antigen serotype, heterologous nucleic acids that encode one or more carrier proteins and/or glycosyltransferases.

In certain embodiments, the host cell used to produce O21 antigen polysaccharides and bioconjugates described herein are engineered to comprise heterologous nucleic acids, e.g., heterologous nucleic acids comprising rfb gene clusters of the O21 antigen serotype, heterologous nucleic acids that encode one or more carrier proteins and/or glycosyltransferases.

In a specific embodiment, heterologous rfb genes, and/or heterologous nucleic acids that encode proteins involved in glycosylation pathways (e.g., prokaryotic and/or eukaryotic glycosylation pathways) can be introduced into the host cells described herein. Such nucleic acids can encode proteins including, but not limited to, oligosaccharyl transferases and/or glycosyltransferases.

A host cell capable of producing a bioconjugate of an E. coli O153 antigen polysaccharide covalently linked to a carrier protein provided herein further comprises a nucleotide sequence of an rfb gene cluster for the E. coli O153 antigen polysaccharide. In certain non-limiting exemplary embodiments, the rfb gene cluster useful for production of the E. coli O153 antigen polysaccharide has the sequence of SEQ ID NO: 3. Another example can be found in GenBank, locus KJ755551.

A host cell capable of producing a bioconjugate of an E. coli O21 antigen polysaccharide covalently linked to a carrier protein provided herein as SEQ ID NO: 4, further comprises a nucleotide sequence of a rfb gene cluster for the E. coli O21 antigen polysaccharide and a nucleotide sequence encoding a UDP-glucose 4-epimerase. In certain non-limiting exemplary embodiments, the rfb gene cluster useful for production of the E. coli O21 antigen polysaccharide has the sequence of SEQ ID NO: 6. Another example can be found in GenBank, locus EU694098. In certain non-limiting exemplary embodiments, the nucleotide sequence encoding the UDP-glucose 4-epimerase useful for production of the E. coli O21 antigen polysaccharide has the sequence of SEQ ID NO: 7. In certain non-limiting embodiments the UDP-glucose 4-epimerase enzyme has the amino acid sequence set forth in SEQ ID NO: 8.

Degenerate nucleic acid sequences encoding the same enzymes as encoded by the provided rfb cluster sequences or UDP-glucose 4-epimerase nucleotide sequence, or sequences that encode enzymes that are at least 80% identical, preferably at least 85%, 90%, 91%, 92%, 93%, 94%, 95%, 96%, 97%, 98%, 99% identical, and that have at least qualitatively the same enzymatic functionality as the respective reference enzymes (i.e. having 100% identity to the encoded enzyme sequences) encoded in the rfb gene cluster or UDP-glucose 4-epimerase, can also be used.

A host cell capable of producing a bioconjugate of an E. coli O153 or O21 antigen polysaccharide further comprises a nucleic acid that encodes an oligosaccharyl transferase. Oligosaccharyl transferases transfer lipid-linked oligosaccharides to asparagine residues of nascent polypeptide chains that comprise an N-glycosylation consensus motif. The nucleic acid that encodes an oligosaccharyl transferase can be native to the host cell, or can be introduced into the host cell using genetic approaches. In preferred embodiments, the oligosaccharyl transferase is heterologous to the host cell. E. coli does not naturally comprise an oligosaccharyl transferase, and hence if E. coli is used as a host cell for production of bioconjugates, a heterologous oligosaccharyl transferase is comprised in such host cell, e.g. upon introduction by genetic engineering. The oligosaccharyl transferase can be from any source known in the art in view of the present disclosure.

In certain embodiments, an alternative to an oligosaccharyl transferase with N-glycosyltransferase activity, such as an O-glycosyltransferase, e.g. as a non-limiting example PgIL, can be used, in conjunction with its own, different, glycosylation consensus sequence in the carrier protein, as for instance described in WO 2016/82597 and WO 2020/120569. Other glycosyltransferases, such as O-glycosyltransferases, can thus also be used as an oligosaccharyltransferase according to the invention.

In certain preferred embodiments, the oligosaccharyl transferase is an oligosaccharyl transferase from Campylobacter. For example, in one embodiment, the oligosaccharyl transferase is an oligosaccharyl transferase from Campylobacter jejuni (i.e., pglB; see, e.g., Wacker et al., 2002, Science 298:1790-1793; see also, e.g., NCBI Gene ID: 3231775, UniProt Accession No. O86154). In another embodiment, the oligosaccharyl transferase is an oligosaccharyl transferase from Campylobacter lari (see, e.g., NCBI Gene ID: 7410986).

In certain embodiments, the oligosaccharyl transferase is PglB oligosaccharyl transferase from Campylobacter jejuni, including the natural (wild-type) protein or any variant thereof, such as those described in International Patent Application Publications WO 2016/107818 and WO 2016/107819. In particular embodiments, the PglB oligosaccharyl transferase comprises SEQ ID NO: 4, or a variant thereof. In certain embodiments one or more endogenous glycosylation consensus sequences in a wild-type PglB have been mutated to avoid PglB autoglycosylation, e.g. SEQ ID NO: 4 comprising the mutation N534Q. Examples of variant PglB oligosaccharyl transferases suitable for use in the recombinant host cells provided herein include the PglB oligosaccharyl transferase of SEQ ID NO: 4 comprising at least one mutation selected from the group consisting of N311V, K482R, D483H, A669V, Y77H, S80R, Q287P, and K289R. In one particular embodiment, a variant PglB oligosaccharyl transferase has SEQ ID NO: 4 comprising the mutation N311V. In a preferred embodiment for production of bioconjugates of E. coli O153 antigen polysaccharide covalently coupled to a carrier protein, a variant PglB oligosaccharyl transferase has SEQ ID NO: 4 comprising the mutations N311V, K482R, D483H, and A669V. In a preferred embodiment for production of bioconjugates of E. coli O21 antigen polysaccharide covalently coupled to a carrier protein, a variant PglB oligosaccharyl transferase has SEQ ID NO: 4 comprising the mutation N311V.

Further variants of PglB that are at least 80%, 85%, 90%, 91%, 92%, 93%, 94%, 95%, 96%, 97%, 98%, 99% identical to SEQ ID NO: 4 and still have oligosaccharyl transferase activity, optionally having one or more of the specific amino acids on the indicated positions herein (e.g. 77Y, 80S, 287Q, 289K, 311N, 482K, 483D, 669A; or 311V; or 311V, 482R, 483H, 669V; or 77H, 80R, 287P, 289R, 311V; or 77H, 311V; etc) can also be used for production of bioconjugates. Preferred PglB enzymes for producing bioconjugates of O-antigens from E. coli serotypes O1, O2, O4, O6, O8, O15, O16, O18, O25, and O75 have been described in WO 2020/191088.

In certain embodiments, additional modifications can be introduced (e.g., using recombinant techniques) into the host cells described herein. For example, host cell nucleic acids (e.g., genes) that encode proteins that form part of a possibly competing or interfering glycosylation pathway (e.g., compete or interfere with one or more heterologous genes involved in glycosylation that are recombinantly introduced into the host cell) can be deleted or modified in the host cell background (genome) in a manner that makes them inactive/dysfunctional (i.e., the host cell nucleic acids that are deleted/modified do not encode a functional protein). In certain embodiments, when nucleic acids are deleted from the genome of the host cells provided herein, they are replaced by a desirable sequence, e.g., a sequence that is useful for production of an O antigen polysaccharide or bioconjugate thereof.

Exemplary genes or gene clusters that can be deleted in host cells (and, in some cases, replaced with other desired nucleic acid sequences) include genes or gene clusters of host cells involved in glycolipid biosynthesis, such as waaL, the lipid A core biosynthesis cluster (waa), galactose cluster (gal), arabinose cluster (ara), colonic acid cluster (wc), capsular polysaccharide cluster, undecaprenol-p biosynthesis genes (e.g. uppS, uppP), und-P recycling genes, metabolic enzymes involved in nucleotide activated sugar biosynthesis, enterobacterial common antigen cluster (eca), and prophage O antigen modification clusters like the gtrABS cluster or regions thereof.

In a specific embodiment, the waaL gene is deleted or functionally inactivated from the genome of a host cell (e.g., recombinant host cell) provided herein. The terms “waaL” and “waaL gene” refer to the O-antigen ligase gene encoding a membrane bound enzyme with an active site located in the periplasm. The encoded enzyme transfers undecaprenylphosphate (UPP)-bound O antigen to the lipid A core, forming lipopolysaccharide. Deletion or disruption of the endogenous waaL gene (e.g., ΔwaaL strains) disrupts transfer of the O-antigen to lipid A, and can instead enhance transfer of the O-antigen to another biomolecule, such as a carrier protein.

In another specific embodiment, one or more of the waaL gene, gtrA gene, gtrB gene, gtrS gene, and the rfb gene cluster is deleted or functionally inactivated from the original genome of a host cell capable of producing a bioconjugate, e.g. of an E. coli O153 antigen polysaccharide or an E. coli O21 antigen polysaccharide.

In certain embodiments, the host cell capable of producing a bioconjugate of an E. coli O153 or O21 antigen polysaccharide further comprises a nucleic acid encoding a carrier protein as previously described herein.

In certain embodiments, each of E. coli O1, O2, O4, O6, O8, O15, O16, O18, O25, O75, O21 and O153 antigen polysaccharides, particularly when part of a bioconjugate, is covalently linked to an asparagine (Asn) residue in the carrier protein wherein the Asn residue is present in a glycosylation site comprising a glycosylation consensus sequence Asn-X-Ser(Thr), wherein X can be any amino acid except Pro. Preferably the Asn residue is present in a glycosylation site comprising a glycosylation consensus sequence Asp (Glu)-X-Asn-Z-Ser(Thr), wherein X and Z are independently selected from any amino acid except Pro (SEQ ID NO: 1). The carrier protein can comprise 1-10 glycosylation sites, preferably 2, 3, or 4 glycosylation sites, most preferably 4 glycosylation sites, each comprising a glycosylation consensus sequence. In a particular embodiment, the carrier protein is EPA-4 carrier protein, for instance EPA-4 carrier protein comprising the amino acid sequence of SEQ ID NO: 2.

In a further aspect, the invention provides for a pharmaceutical composition comprising a pharmaceutically acceptable carrier and/or excipient and the composition as described herein. As used herein, the term “pharmaceutically acceptable” means approved by a regulatory agency of a Federal or a state government or listed in the U.S. Pharmacopeia or other generally recognized pharmacopeia for use in animals, and more particularly in humans. The term “carrier,” as used herein in the context of a pharmaceutically acceptable carrier, refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in textbooks known to the skilled person in the field of pharmaceutical formulations.

In certain embodiments, the compositions described herein (e.g., the immunogenic compositions) comprise, or are administered in combination with, an adjuvant. In some embodiments, the term “adjuvant” refers to a compound that when administered in conjunction with or as part of a composition described herein augments, enhances and/or boosts the immune response to the (bio) conjugates, but when the adjuvant compound is administered alone does not generate an immune response to the (bio) conjugate. Examples of suitable adjuvants include, but are not limited to, aluminum salts (alum) (such as aluminum hydroxide, aluminum phosphate, aluminum sulfate and aluminum oxide, including nanoparticles comprising alum or nanoalum formulations), calcium phosphate, monophosphoryl lipid A (MPL) or 3-de-O-acylated monophosphoryl lipid A (3D-MPL) (see e.g., GB2220211, EP0971739, EP1194166, U.S. Pat. No. 6,491,919), AS01, AS02, AS03 and AS04 (all GlaxoSmithKline; see e.g. EP1126876, U.S. Pat. No. 7,357,936 for AS04, EP0671948, EP0761231, U.S. Pat. No. 5,750,110 for AS02), MF59 (Novartis), imidazopyridine compounds (see WO2007/109812), imidazoquinoxaline compounds (see WO2007/109813), delta-inulin, STING-activating synthetic cyclic-di-nucleotides (e.g. US20150056224), combinations of lecithin and carbomer homopolymers (e.g. U.S. Pat. No. 6,676,958), and saponins, such as QuilA and QS21, Matrix M, Iscoms, Iscomatrix, etc, optionally in combination with QS7. In some embodiments, the adjuvant is Freund's adjuvant (complete or incomplete). Other adjuvants are oil in water emulsions (such as squalene or peanut oil), optionally in combination with immune stimulants, such as monophosphoryl lipid A. Another adjuvant is CpG. Further examples of adjuvants are liposomes containing immune stimulants such as MPL and QS21 such as in AS01E and AS01B. Other examples of adjuvants are imidazoquinolines (such as imiquimod and R848. In certain embodiments, the adjuvant contains a toll-like receptor 4 (TLR4) agonist. TLR4 agonists are well known in the art, see e.g. Ireton G C and. In certain embodiments, the adjuvant comprises a TLR4 agonist comprising lipid A, or an analog or derivative thereof, such as MPL, 3D-MPL, RC529, PET-lipid A, GLA (glycopyranosyl lipid adjuvant, a synthetic disaccharide glycolipid), SLA, which describes a structure-function approach to optimize TLR4 ligands for human vaccines), PHAD (phosphorylated hexaacyl disaccharide), 3D-PHAD (the structure of which is the same as that of GLA), 3D-(6-acyl)-PHAD (3D (6A)-PHAD) (PHAD, 3D-PHAD, and 3D (6A) PHAD are synthetic lipid A variants, see e.g. avantilipids.com/divisions/adjuvants, which also provide structures of these molecules), E6020 (CAS Number 287180-63-6), ONO4007, OM-174, and the like.

In certain preferred embodiments, the compositions described herein do not comprise an adjuvant besides the bioconjugates, and/or are not administered in combination with an adjuvant besides the bioconjugates (in case the bioconjugates would comprise some intrinsic adjuvant properties, these would be disregarded and no extrinsic adjuvant would be added in these embodiments).

The skilled person is aware that various formulations can be used for compositions of the invention. In one embodiment, a composition of the invention comprises the (bio) conjugates as described herein in a Tris-buffered saline (TBS) pH 7.4 (e.g. containing Tris, NaCl and KCl, e.g. at 25 mM, 137 mM and 2.7 mM, respectively). In other embodiments, the compositions of the invention comprise (bio) conjugates as described herein in about 10 mM KH2PO4/Na2HPO4 buffer at pH of about 7.0, about 5% (w/V) sorbitol, about 10 mM methionine, and about 0.02% (w/v) polysorbate 80. In other embodiments, the compositions of the invention comprise (bio) conjugates as described herein in about 10 mM KH2PO4/Na2HPO4 buffer at pH of about 7.0, about 8% (w/v) sucrose, about 1 mM EDTA, and about 0.02% (w/v) polysorbate 80 (see e.g. WO 2018/077853 for suitable buffers for bioconjugates of E. coli O-antigens covalently bound to EPA carrier protein). In other embodiments, the compositions of the invention comprise (bio) conjugates as described herein in about 5 mM succinate/0.9% NaCl, pH 6.0.

Typically the compositions of the invention can be prepared by first obtaining individual glycoconjugates for each of the E. coli O-antigen polysaccharides as described herein by independently covalently linking these O-antigen polysaccharides to a carrier protein e.g. by chemical conjugation or bioconjugation, and subsequently mixing the individual glycoconjugates in amounts and ratios as described herein to obtain compositions according to the invention. It is therefore a further aspect of the invention to provide methods to prepare compositions according to the invention, comprising providing each of the required O-antigen conjugates (e.g., by obtaining or manufacturing these, e.g., in the form of drug substances), and mixing them in the desired ratios and/or amounts to obtain a composition of the invention (e.g., a multivalent E. coli, particularly ExPEC, vaccine composition, sometimes referred to as drug product).

Accordingly in one aspect, the invention also provides a method for preparing a composition of the invention by producing a bioconjugate of an E. coli O153 antigen polysaccharide covalently linked to a carrier protein, the method comprising culturing a recombinant prokaryotic host cell of the invention to produce the bioconjugate of an E. coli O153 of the invention, and mixing said bioconjugate with further glycoconjugates, preferably bioconjugates (e.g. bioconjugates of E. coli O1, O2, O4, O6, O8, O15, O16, O18, O25, O75, and/or O21 antigen polysaccharides each independently covalently linked to a carrier protein) to obtain a composition according to the invention as described herein. In one aspect, the invention also provides a method for preparing a composition of the invention by producing a bioconjugate of an E. coli O21 antigen polysaccharide covalently linked to a carrier protein, the method comprising culturing a recombinant prokaryotic host cell of the invention to produce the bioconjugate of an E. coli O21 of the invention, and mixing said bioconjugate with further glycoconjugates, preferably bioconjugates (e.g. bioconjugates of E. coli O1, O2, O4, O6, O8, O15, O16, O18, O25, O75, and/or O153 antigen polysaccharides each independently covalently linked to a carrier protein) to obtain a composition according to the invention as described herein.

(Bio) conjugates and compositions provided herein can be used to induce antibodies against an E. coli O-antigen in a subject, or to vaccinate a subject against E. coli. The methods of inducing an immune response in a subject described herein result in vaccination of the subject against infection or resulting disease by the E. coli strains from serotypes whose O-antigens are present in the composition(s).

Accordingly in a further aspect, the invention provides for a method of inducing an immune response to E. coli, preferably extra-intestinal pathogenic E. coli (ExPEC), in a subject wherein the method comprises administering to the subject the composition as described herein or the pharmaceutical composition as described herein.

In certain aspects, provided herein is a composition as described herein for use in a method of inducing an immune response to E. coli, preferably ExPEC, in a subject. In certain aspects, provided herein is use of a composition as described herein in the manufacture of a medicament for inducing an immune response to E. coli, preferably ExPEC, in a subject.

In certain embodiments, the subject is human. In some embodiments, the subject is a human having or at risk of having an ExPEC infection or an invasive ExPEC disease. In one embodiment, the subject has an E. coli (e.g., ExPEC) infection at the time of administration. In a preferred embodiment, the subject does not have an E. coli (e.g., ExPEC) infection or invasive ExPEC disease at the time of administration.

In certain embodiments according to the methods of the invention, the immune response limits the severity of or prevents an invasive ExPEC disease in the subject, preferably wherein the invasive ExPEC disease comprises sepsis and/or bacteremia.

In certain embodiments, the immune response induced in a subject following administration of a composition described herein is effective to prevent or reduce a symptom resulting from an ExPEC infection, preferably in at least 30%, more preferably at least 40%, such as at least 50%, of the subjects administered with the composition. Symptoms of ExPEC infection may vary depending on the nature of the infection and may include, but are not limited to: dysuria, increased urinary frequency or urgency, pyuria, hematuria, back pain, pelvic pain, pain while urinating, fever, chills, and/or nausea (e.g., in subjects having a urinary tract infection caused by ExPEC); high fever, headache, stiff neck, nausea, vomiting, seizures, sleepiness, and/or light sensitivity (e.g., in subjects having meningitis caused by ExPEC); fever, increased heart rate, increased respiratory rate, decreased urine output, decreased platelet count, abdominal pain, difficulty breathing, and/or abnormal heart function (e.g., in subjects having sepsis caused by ExPEC).

### EXAMPLES

The following examples of the invention are to further illustrate the nature of the invention. It should be understood that the following examples do not limit the invention and the scope of the invention is to be determined by the appended claims.

### Example 1: Production of Bioconjugates of E. coli O153 Antigen Polysaccharide Linked to EPA Carrier Protein

O153 is an E. coli serotype that has been observed in more than 1% of ExPEC blood culture isolates from patient populations across various regions of the world and appeared present with a relatively high prevalence of over 8% in such isolates from South America (Weerdenburg et al, 2022, Clin Infect Dis., doi: 10.1093/cid/ciac421). It could therefore be useful to include antigens that could protect against the E. coli O153 serotype in vaccine compositions, e.g. in the form of a conjugate of the O-antigen linked to a carrier protein. This example describes the production of a bioconjugate of E. coli O153 antigen polysaccharide linked to EPA as a carrier protein.

An E. coli O153 blood isolate was selected as O153 donor (collected in the Netherlands, obtained from Utrecht University Medical Center) for the rfb gene cluster. As parent strain, E. coli K12 strain W3110 was used. This strain can for instance be obtained from the E. coli Genetic Stock Center (Yale University, New Haven (CT), USA, product number CGSC #4474). The genomic sequence was published (PMID: 16738553).

The parent E. coli K12 strain W3110 was modified by replacing the E. coli W3110 chromosomal rfb cluster with the rfb cluster of a clinical isolate E. coli O153 strain (the rfb cluster having SEQ ID NO: 3) by previously described methods (see e.g. WO 2020/191082). Additionally, the O-antigen ligase waaL was deleted to avoid transfer of the O-antigen to lipid A, and thereby enhance transfer of the O-antigen to the carrier protein. The chromosomal gtrABS genes were disrupted by homologous recombination to prevent addition of branching glucose to the O153 antigen.

To confirm the production of O153 antigen the modified strain was grown in LB media w/o antibiotics at 37° C. o/n. After harvest, the cell pellet was resuspended in Laemmli buffer and proteins were digested with Protease K. LPS/LLOs were separated by SDS-PAGE and O153-antigen was detected by Western blot using an O153 specific antiserum. The structure of the O153-antigen was confirmed by MALDIMS/MSMS.

A plasmid encoding the gene for genetically detoxified Pseudomonas aeruginosa exotoxin (EPA) with two glycosylation sites (EPA-2) or encoding the gene for EPA with four glycosylation sites (EPA-4; sequence provided as SEQ ID was introduced. NO: 2) Several plasmids encoding oligosaccharyltransferase Campylobacter jejuni PglB were tested in a separate experiment and it was determined that the plasmid encoding the oligosaccharyltransferase Campylobacter jejuni PalB^{N311V.K482R,D483H,A669V} (which is a PglB variant that has the amino acid sequence depicted in SEQ ID NO: 4 with amino acid substitutions N311V, K482R, D483H and A669V) gave optimal results among the variants tested, which was an unpredictable result. Hence, the plasmid encoding this optimal PglB variant was selected and introduced into the modified host cell to create the production strain for production of bioconjugates of E. coli O153 antigen polysaccharide linked to EPA carrier protein.

To confirm the production of O153 bioconjugate the production strain was grown in TB media supplemented with phosphate buffer, MgCl₂ and antibiotics at 37° C. At an optical density at 600 nm of approximately 4.0, IPTG and arabinose were added to induce the production of bioconjugate. After o/n growth the cells were harvested and the bioconjugate was released by osmotic shock. The O153 bioconjugate was separated by SDS-PAGE and detected by Western blot using an O153 and EPA specific antiserum.

Production in bioreactors and purification of the bioconjugate was performed using known methods (see e.g. WO 2020/191082, WO 2022/214620).

### Example 2: Production of Bioconjugates of E. coli O21 Antigen Polysaccharide Linked to EPA Carrier Protein

O21 is an E. coli serotype that has been observed in more than 1% of ExPEC blood culture isolates from patient populations in North America (Weerdenburg et al, 2022, Clin Infect Dis., doi: 10.1093/cid/ciac421). It could therefore be useful to include antigens that could protect against the E. coli O21 serotype in vaccine compositions, e.g. in the form of a conjugate of the O-antigen linked to a carrier protein. This example describes the production of a bioconjugate of E. coli O21 antigen polysaccharide linked to EPA as a carrier protein.

An E. coli O21 blood isolate was selected as O21 donor (collected in the Netherlands, obtained from Utrecht University Medical Center) for the rfb gene cluster and the gne gene (encoding UDP-glucose 4-epimerase). As parent strain, E. coli K12 strain W3110 was used. This strain can for instance be obtained from the E. coli Genetic Stock Center (Yale University, New Haven (CT), USA, product number CGSC #4474). The genomic sequence was published (PMID: 16738553).

The parent E. coli K12 strain W3110 was modified by replacing the E. coli W3110 chromosomal rfb cluster with the rfb cluster of a clinical isolate E. coli O21 strain (the rfb cluster having SEQ ID NO: 6) by previously described methods (see e.g. WO 2020/191082) and by introducing the gne gene, in the form of a nucleotide sequence (SEQ ID NO: 7) encoding UDP-glucose 4-epimerase (having SEQ ID NO: 8). Additionally, the O-antigen ligase waaL was deleted to avoid transfer of the O-antigen to lipid A, and thereby enhance transfer of the O-antigen to the carrier protein. The chromosomal gtrABS genes were disrupted by homologous recombination to prevent addition of branching glucose to the O21 antigen.

To confirm the production of O21 antigen the modified strain was grown in LB media w/o antibiotics at 37° C. o/n. After harvest, the cell pellet was resuspended in Laemmli buffer and proteins were digested with Protease K. LPS/LLOs were separated by SDS-PAGE and O21-antigen was detected by Western blot using an O21 specific antiserum. The structure of the O21-antigen was confirmed by MALDIMS/MSMS.

A plasmid encoding the gene for genetically detoxified Pseudomonas aeruginosa exotoxin with two glycosylation sites (EPA-2) or encoding the gene for genetically detoxified Pseudomonas aeruginosa exotoxin with four glycosylation sites (EPA-4) were introduced. Several plasmids encoding oligosaccharyltransferase Campylobacter jejuni PglB were tested in a separate experiment and it was determined that the plasmid encoding the oligosaccharyltransferase Campylobacter jejuni PglB^{N311V} (which is a PglB variant that has the amino acid sequence depicted in SEQ ID NO: 4 with amino acid substitution N311V) gave optimal results among the variants tested, which confirmed that the optimal PglB for preparing bioconjugates differs between E. coli serotypes in an unpredictable manner (see previous example and WO 2020/191088). Hence, this optimal PglB variant was introduced into the modified host cell to create the production strain for production of bioconjugates of E. coli O21 antigen polysaccharide linked to EPA carrier protein.

To confirm the production of O21 bioconjugate the production strain was grown in TB media supplemented with phosphate buffer, MgCl₂ and antibiotics at 37° C. At an optical density at 600 nm of approximately 4.0, IPTG and arabinose were added to induce the production of bioconjugate. After o/n growth the cells were harvested and the bioconjugate was released by osmotic shock. The O21 bioconjugate was separated by SDS-PAGE and detected by Western blot using an O21 and EPA specific antiserum.

Production in bioreactors and purification of the bioconjugate was performed using known methods (see e.g. WO 2020/191082, WO 2022/214620).

### Example 3: Immunogenicity of O153 Bioconjugate

### Experimental Design

Female Sprague Dawley rats (5-6 weeks old) were immunized intramuscularly with 0.04, 0.40 or 4.00 μg of O153-EPA bioconjugate at days 0, 14 and 28. The control group received only formulation buffer. Blood samples were drawn pre-immunization (day 0) and post-immunization (day 14 and 42). Blood was processed and serum samples were stored at −20° C. Total IgG Ab responses in serum were measured by ELISA (as previously described). In brief, ELISA plates were coated with O153-LPS, blocked with skimmed milk and incubated with rat serum samples. Subsequently, plates were incubated with HRP-labeled goat-anti-rat IgG and OD450 was measured upon development with TMB substrate. EC50 titers, defined as half maximal effective concentration, were calculated based on duplicate 12-step titration curves that were plotted in a 4PL nonlinear regression model.

### Results

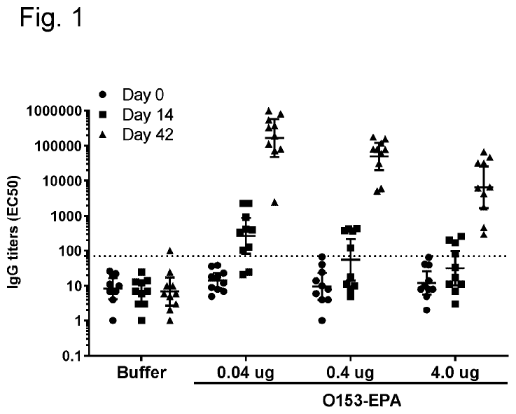

Immunization with 0.04 μg, 0.40 μg and 4.00 μg of O153-EPA bioconjugate per dose induced a significant increase in the levels of IgG antibodies at day 42 compared to formulation buffer (FIG. 1). For rats immunized with 0.04 μg conjugate per dose, a significant increase in Ab titers was also observed at day 14 compared to formulation buffer (FIG. 1). In addition, Ab levels induced by 0.04 μg, 0.40 μg and 4.00 μg of conjugate were significantly increased at day 42 as compared to those detected at day 0 and day 14 post-immunization (FIG. 1). The groups that received 0.04 μg or 4.00 μg conjugate per dose also showed a significant increase in antibody (Ab) levels at day 14 compared to day 0, indicating that a single dose of 0.04 μg or 4.00 μg of O153-EPA conjugate is able to induce a significant increase in IgG titers (FIG. 1). The significant increase in IgG titers between day 14 and day 42 for all three concentrations of bioconjugate shows that the O153-EPA conjugate is able to boost the antibody responses. Highest Ab titers were detected in the group of animals immunized with 0.04 μg/dose, followed by 0.4 μg/dose. Across-dose analysis confirmed these findings, suggesting that better Ab responses were obtained with the lowest doses of O153-EPA conjugate.

This example demonstrates that a bioconjugate wherein the E. coli O153 antigen polysaccharide is covalently linked to a carrier protein is immunogenic.

### Example 4: Immunogenicity of O21 Bioconjugate

### Experimental Design

Female Sprague Dawley rats (5-6 weeks old) were immunized intramuscularly with 0.04, 0.40 or 4.00 μg of O21-EPA bioconjugate at days 0, 14 and 28. The control group received only formulation buffer. Blood samples were drawn pre-immunization (day 0) and post-immunization (day 14 and 42). Blood was processed and serum samples were stored at −20° C. Total IgG Ab responses in serum were measured by ELISA (as previously described). In brief, ELISA plates were coated with O21-LPS, blocked with skimmed milk and incubated with rat serum samples. Subsequently, plates were incubated with HRP-labeled goat-anti-rat IgG and OD450 was measured upon development with TMB substrate. EC50 titers, defined as half maximal effective concentration, were calculated based on duplicate 12-step titration curves that were plotted in a 4PL nonlinear regression model.

### Results

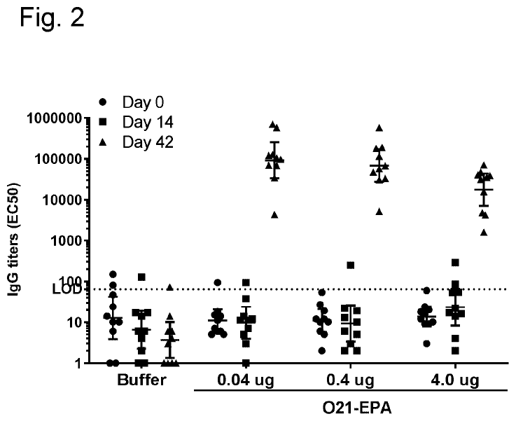

Immunization with 0.04 μg, 0.40 μg and 4.00 μg of O21-EPA bioconjugate per dose induced a significant increase in the levels of IgG antibodies at day 42 compared to formulation buffer (FIG. 2). In addition, Ab levels induced by 0.04 μg, 0.40 μg and 4.00 μg of conjugate were significantly increased at day 42 as compared to those detected at baseline (day 0) and at day 14 post-immunization (FIG. 2). The significant increase in IgG titers between day 14 and day 42, for all three concentrations of bioconjugate shows that the O21-EPA conjugate is able to boost the antibody responses. For the group that received formulation buffer, a significant reduction in Ab levels was observed at day 42 compared to day 0 (FIG. 2), however, most samples of this group did not show a 4PL fitting. Highest titers were detected upon immunization with 0.04 μg/dose. Highest Ab titers were detected in the group of animals immunized with 0.04 μg/dose. Across-dose analysis confirmed this finding, suggesting that better Ab responses were obtained with the lowest dose of O21-EPA conjugate.

This example demonstrates that a bioconjugate wherein the E. coli O21 antigen polysaccharide is covalently linked to a carrier protein is immunogenic.

### Example 5: Opsonophagocytic Killing Mediated by Antibodies Induced by the O153-EPA or the O21-EPA Bioconjugate

### Experimental Design

Functional activity of serum antibodies was measured by opsonophagocytic killing assay (OPKA; protocol as previously described). In brief, serum samples were heat-inactivated at 56° C. for 30 min and diluted 1:10 (starting dilution), followed by an 8-step 3-fold serial dilution. All serum samples were tested in duplicate. Serum was incubated with bacteria (~103 CFU) for 30 min. at RT, and subsequently, complement and HL60 cells were added and incubated for 1 hour at 37° C. The final reaction mixture was spotted onto agar plates and incubated for 15-16 hours at 33° C. The number of bacterial colonies on the plates was counted and opsonization index (OI) values, defined as the serum dilution that kills 50% of bacteria, were calculated using the Opsotiter3 Excel-based program (Department of Microbiology, University of Alabama, Birmingham). Due to the complexity of the assay, antibodies functionality was assessed only for samples of rats immunized with formulation buffer or 4.00 μg/dose O153-EPA/O21-EPA conjugates, respectively, at day 42.

### Results

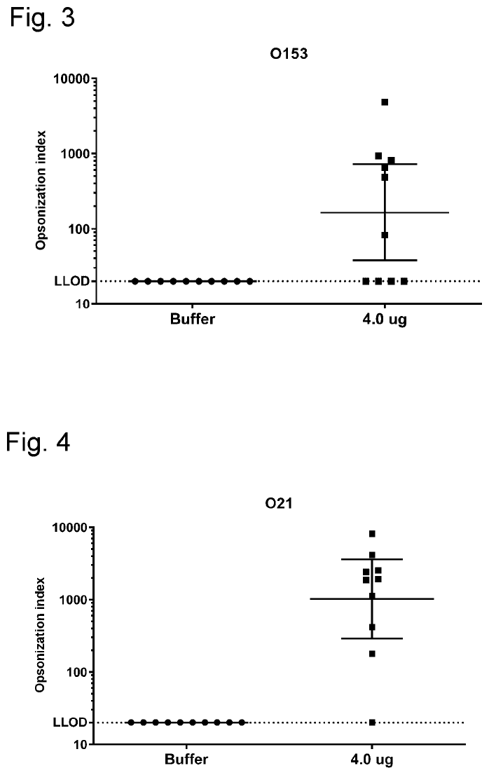

A significant increase in E. coli OI was observed with serum samples from animals immunized with O153-EPA conjugate (FIG. 3) and from animals immunized with O21-EPA conjugate (FIG. 4) when compared to formulation buffer These results show that O153-induced antibodies and O21-induced antibodies induced by administration of O153-EPA and O21-EPA conjugates respectively, are functional and able to mediate E. coli killing.

### Example 6: Production of Composition Comprising 12 E. coli O-Antigen Conjugates

Preparation of bioconjugates for E. coli O1 (O1A), O2, O4 (O4A), O6 (O6A), O8, O15, O16, O18, O25 (O25B), and O75 has been described in detail in WO2020/191082, preparation of bioconjugates for E. coli O18 has been described in detail in PCT/IB2022/053013, and preparation of conjugate compositions with E. coli O75 and other E. coli O-antigen conjugates has been described in detail in WO2022/058945. Production of E. coli O153 and E. coli O21 bioconjugates was done as described herein. The 12 conjugates were blended together to provide for an immunogenic composition described in Table 2:

TABLE 2
12-valent composition of E. coli bioconjugates

| | | ExPEC12V | ExPEC12V |
|---|---|---|---|
| | | Composition 1 | Composition 2 |
| | | (amount in ug | (amount in ug |
| Conjugate | Carrier | polysaccharide/dose) | polysaccharide/dose) |
| O1A | EPA-4 | 4.0 | 0.4 |
| O2 | EPA-4 | 4.0 | 0.4 |
| O4A | EPA-2 | 4.0 | 0.4 |
| O6A | EPA-4 | 4.0 | 0.4 |
| O8 | EPA-2 | 4.0 | 0.4 |
| O15 | EPA-2 | 4.0 | 0.4 |
| O16 | EPA-2 | 4.0 | 0.4 |
| O18 | EPA-2 | 4.0 | 0.4 |
| O25B | EPA-4 | 8.0 | 0.8 |
| O75 | EPA-2 | 4.0 | 0.4 |
| O21 | EPA-2 | 4.0 | 0.4 |
| O153 | EPA-2 | 4.0 | 0.4 |

A composition comprising the 12 bioconjugates is referred to herein as ‘ExPEC12V’.

### Example 7: Induction of Immune Response by ExPEC12V Compositions

### Experimental Design

Female Sprague Dawley rats (5-6 weeks old) were immunized intramuscularly with ExPEC12V vaccine at days 0, 14 and 28. Animals were divided in 3 different treatment groups: Group 1:4.0 μg of each polysaccharide (PS)/dose (except O25B, which was given at 8.0 μg PS/dose); Group 2: 0.4 μg of each PS/dose (except O25B, which was given at 0.8 μg PS/dose); Group 3: control group that received formulation buffer. Blood samples were drawn pre-immunization (day 0) and post-immunization (day 28 and 42). Levels of total IgG in serum was measured by ELISA (according to industry standards). In brief, ELISA plates were coated with O1A, O2, O4, O6A, O8, O15, O16, O18, O21, O25B, O75, O21 or O153 LPS, blocked with skimmed milk and incubated with rat serum samples. Subsequently, plates were incubated with HRP-labeled goat-anti-rat IgG and OD450 was measured upon development with TMB substrate. EC50 titers, defined as half maximal effective concentration, were calculated based on duplicate 12-step titration curves that were plotted in a 4PL nonlinear regression model.

### Results

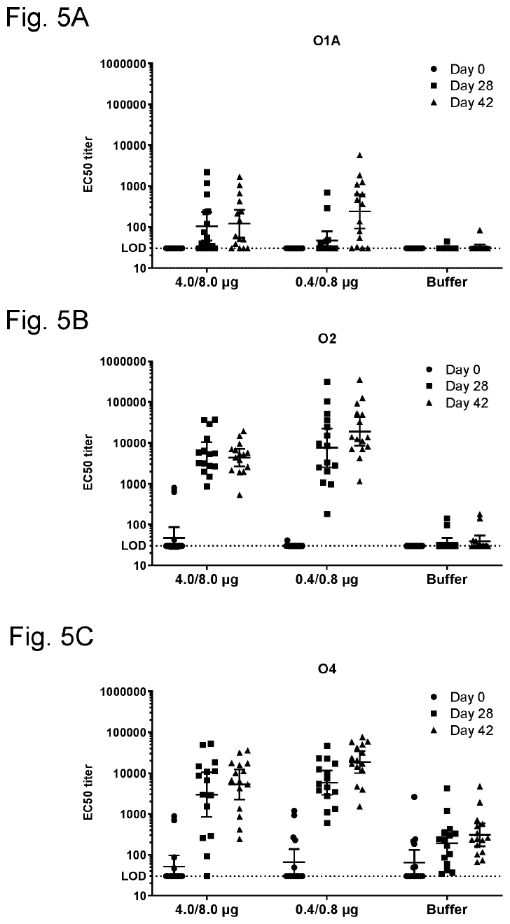

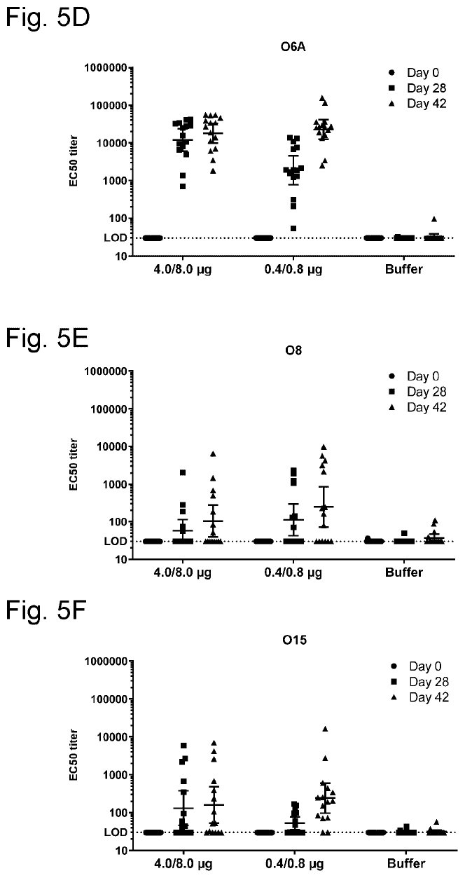

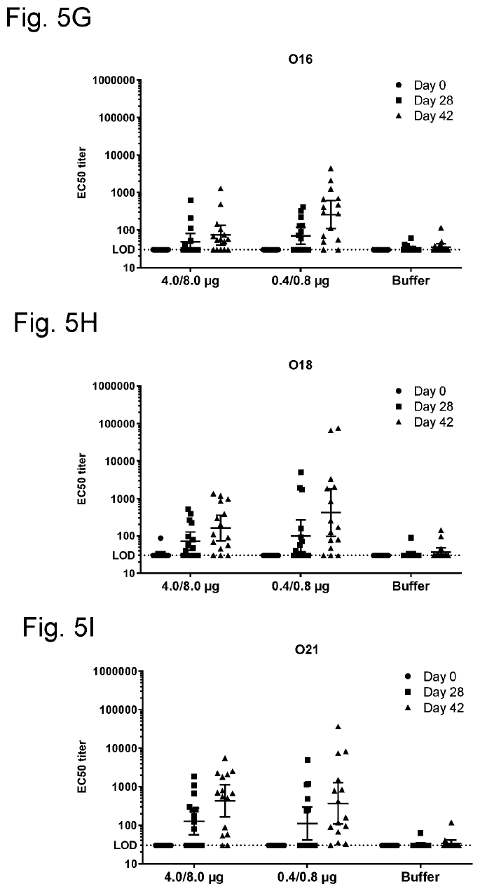

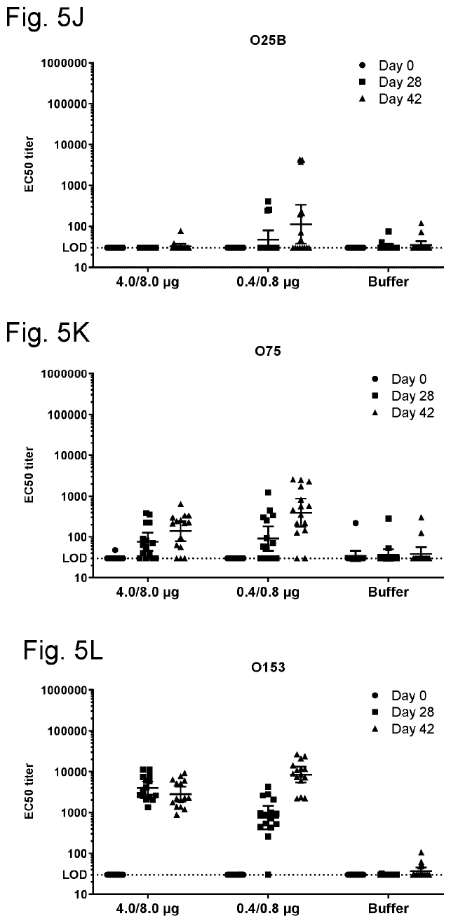

Upon immunization with 0.4/0.8 μg PS/dose of ExPEC12V, significantly higher IgG antibody titers were observed at day 42 compared to formulation buffer. In addition, Ab levels induced by 0.4/0.8 μg PS/dose of ExPEC12V were significantly increased at day 42 as compared to those detected at baseline (day 0) for all conjugates tested (FIG. 5). For most conjugates, Ab levels were significantly increased already at day 28 compared to day 0, except for O1A, O21 and O25B. Furthermore, a significant increase in Ab levels at day 42 compared to day 28 was observed for all conjugates at 0.4/0.8 μg PS/dose ExPEC12V, indicating that the boost dose of ExPEC12V given at day 28 is able to increase the Ab responses (FIG. 5).

Comparison of the groups that received 0.4/0.8 μg PS/dose or 4/8 μg PS/dose showed that immunization with 0.4/0.8 μg PS/dose induced highest Ab responses for O2, O4 and O153 conjugates when compared to 4.0/8.0 μg PS/dose.

This example demonstrates that E. coli O21 and O153 conjugates are also immunogenic in a multivalent vaccine composition comprising several other bioconjugates of E. coli O-antigens representing different serotypes, as well as showing that the other bioconjugates that previously were shown to be immunogenic in a 10-valent composition (see e.g. WO 2020/191082) remain immunogenic when bioconjugates of E. coli O21 and O153 are added to generate a 12-valent composition.

### Example 8: Opsonophagocytic Killing, Mediated by Abs Induced by the ExPEC12V Vaccine

### Experimental Design

Functional activity of serum Abs was measured by monoplex opsonophagocytic killing assay (OPKA) for O1A, O2, O4, O8, O15, O16, O18, O21, O75 and O153 or multiplex opsonophagocytic killing assay (MOPA) for O6A and O25B. OPKA protocol was performed as described in example 5. For MOPA serum samples were heat-inactivated at 56° C. for 30 min and diluted 1:10 (starting dilution), followed by an 8-step 3-fold serial dilution. Serum was incubated with bacteria for 30 min. at RT. Subsequently, complement and HL60 cells were added and incubated for 1 hour at 37° C. The final reaction mixture was spotted onto agar plates and incubated for 15-16 hours at 33° C. and plates containing distinct types of antibiotics were used to select for a specific antibiotic-resistant strain in the reaction mixture.

The number of bacterial colonies on the plates was counted and opsonization index (OI) values, defined as the serum dilution that kills 50% of bacteria, were calculated using the Opsotiter3 Excel-based program (Department of Microbiology, University of Alabama, Birmingham). Due to the complexity of the assay, Ab functionality was assessed only with samples from day 42 post-immunization.

### Results

Functional activity of serum Abs was measured by OPKA for each serotype included in the ExPEC12V vaccine. Upon immunization with 0.4/0.8 μg/PS/dose of ExPEC12V, significant higher opsonophagocytic titers were observed for E. coli strains O4, O8, O15, O18, O25B and O153 at day 42 compared to formulation buffer (Table 3). For O1A, killing of bacteria was observed in only 2 out of 15 animals, for O2, killing of bacteria was observed in 5 out of 15 animals, and for O21, killing was observed in 4 out of 15 animals that received 0.4/0.8 μg/PS/dose ExPEC12V. For O6A, serum of 13 out of 15 animals mediated killing, whereas in the group that received buffer, serum of only 7 out of 15 animals mediated killing, however, this difference did not reach statistical significance. For O16 and O75, killing was observed in 15 out of 15 animals in the groups that were immunized with 0.4/0.8 μg/PS/dose of the ExPEC12V vaccine, however, as killing was also observed in the buffer group, no significant difference in opsonic titers could be detected for these serotypes. Upon immunization with 4/8 μg/PS/dose of ExPEC12V, significant higher opsonophagocytic titers were observed only for E. coli strains O4 and O15 at day 42 compared to formulation buffer. For O8, killing was observed in only 6 out of 15 animals, for O21 killing was observed in 3 out of 15 animals, and for O25B killing was observed in 4 out of 15 animals that received 4/8 μg/PS/dose of ExPEC12V. For O6A, killing was observed in 7 out of 15 animals and for O16 and O75, killing was observed in 15 out of 15 animals in the group that was immunized with 4/8 μg/PS/dose of the ExPEC12V vaccine, however, the number of animals for which killing was detected was similar in the buffer group.

An overview of GMT values of the opsonization index measured by OPKA for each serotype included in the ExPEC12V vaccine is shown in Table 3.

TABLE 3
Overview of GMT values of the opsonization
index (OI) for the indicated serotypes
GMT (OI)

| serotype | 4/8 μg | 0.4/0.8 μg | buffer |
|---|---|---|---|
| O1A | 20 | 35 | 20 |
| O2 | 21 | 29 | 20 |
| O4 | 1475 | 4887 | 429 |
| O6A | 48 | 95 | 45 |
| O8 | 41 | 130 | 21 |
| O15 | 350 | 459 | 34 |
| O16 | 687 | 692 | 604 |
| O18 | 214 | 293 | 186 |
| O21 | 24 | 34 | 21 |
| O25B | 35 | 73 | 24 |
| O75 | 834 | 793 | 680 |
| O153 | 20 | 936 | 20 |

Overall, the ExPEC12V composition was able to induce functional antibodies against several of the serotypes for which O-antigens were present in the composition, while for some serotypes no significant induction of functional antibodies could be observed, which is not uncommon in such studies performed in rats, and usually the result of high background levels of functional antibodies against the respective serotype, i.e. more relating to the assay and used E. coli strains in the assay rather than indicating that functional antibodies against such serotypes would not be induced by the composition, and typically functional antibodies can be detected when tested in other animals and/or humans.

SEQUENCES

SEQ ID NO: 1 (Optimized glycosylation consensus sequence)
Asp(Glu)-X-Asn-Z-Ser(Thr), wherein X and Z are independently selected from any
amino acid except Pro
SEQ ID NO: 2 (EPA carrier protein comprising 4 glycosylation consensus sequences (EPA-4)
G SGGGDQNATG SGGGKLAEEA FDLWNECAKA CVLDLKDGVR SSRMSVDPAI ADTNGQGVLH
YSMVLEGGND ALKLAIDNAL SITSDGLTIR LEGGVEPNKP VRYSYTRQAR GSWSLNWLVP IGHEKPSNIK
VFIHELNAGN QLSHMSPIYT IEMGDELLAK LARDATFFVR AHESNEMQPT LAISHAGVSV VMAQAQPRRE
KRWSEWASGK VLCLLDPLDG VYNYLAQQRC NLDDTWEGKI YRVLAGNPAK HDLDIKDNNN STPTVISHRL
HFPEGGSLAA LTAHQACHLP LEAFTRHRQP RGWEQLEQCG YPVQRLVALY LAARLSWNQV DQVIRNALAS
PGSGGDLGEA IREQPEQARL ALTLAAAESE RFVRQGTGND EAGAASADVV SLTCPVAKDQ NRTKGECAGP
ADSGDALLER NYPTGAEFLG DGGDVSFSTR GTQNWTVERL LQAHRQLEER GYVFVGYHGT FLEAAQSIVF
GGVRARSQDL DAIWRGFYIA GDPALAYGYA QDQEPDARGR IRNGALLRVY VPRWSLPGFY RTGLTLAAPE
AAGEVERLIG HPLPLRLDAI TGPEEEGGRV TILGWPLAER TVVIPSAIPT DPRNVGGDLD PSSIPDKEQA
ISALPDYASQ PGKPPREDLK LGSGGGDQNA T
SEQ ID NO: 3 rfb cluster E. coli O153
ATGACGAATTTAAAAGCAGTTATTCCTGTAGCGGGTCTCGGGATGCATATGTTGCCTGCCACTAAGGCGATACCCAA
AGAGATGCTACCAATCGTCGACAAGCCAATGATTCAGTACATTGTTGACGAGATTGTGGCTGCAGGGATCAAAGAAA
TCCTCCTGGTAACTCACGCGTCCAAGAACGCGGTCGAAAACCACTTCGACACCTCTTATGAGTTAGAATCACTCCTT
GAGCAGCGCGTGAAGCGTCAACTGCTGGCGGAAGTACAGTCCATCTGTCCGCCGGGCGTGACCATTATGAACGTGCG
TCAGGGCGAACCTTTAGGTTTAGGCCACTCCATTTTGTGTGCGCGACCTGCCATTGGTGACAACCCATTTGTCGTGG
TACTGCCAGACGTTGTGATCGACGATGCCAGCGCCGACCCGCTACGTTACAACCTTGCTGCCATGATTGCACGTTTC
AACGAAACGGGCCGCAGCCAGGTGCTGGCAAAACGTATGCCGGGTGACCTCTCTGAATACTCCGTCATCCAGACTAA
AGAGCCGCTGGACCGTGAGGGTAAAGTCAGCCGCATTGTTGAATTTATCGAAAAACCGGATCAGCCGCAGACGOTGG
ACTCAGACATCATGGCCGTAGGTCGCTATGTGCTTTCTGCCGATATTTGGCCGGAACTGGAACGTACTCAGCCTGGT
GCATGGGGACGTATTCAGCTGACTGATGCTATTGCCGAGCTGGCGAAAAAACAATCCGTTGATGCAATGCTGATGAC
CGGCGACAGTTACGACTGCGGCAAAAAAATGGGCTATATGCAGGCGTTTGTGAAGTATGGCCTACGCAACCTGAAAG
AAGGGGCGAAGTTCCGTAAAGGTATTGAGAAGCTGTTAAGCGAATAATGAAAATCTGACCGGATGTAACGGTTGATA
AGAAAATTATAACGGCAGTGAAAATTCGCAGCAAAAGTAATTTGTTGCGAATCTTCCTGCCGTTGTTTTATATAAAC
CATCAGAATAACAACGAGTTAGCAGTAGGGTTTTATTCAAAGTTTTCCAGGATTTTCCTTGTTTCCAGAGCGGATTG
GTAAGACAATTAGCGTTTGAATTTTTCGGGTTTAGCGCGAGTGGGTAACGCTCGTCACATCATAGGCATGCATGCAG
TGCTCTGGTAGCTGTAAAGCCAGGGGCGGTAGCGTGCATTAATACCTCTATTAATCAAACTGAGAGCCGCTTATTTC
ACAGCATGCTCTGAAGTAATATGGAATAAATTAAGCTAGCGATCGCATGAAAAATAAGTGCCCACGAATTGCAGTAA
TAATGTCGGTTTATATTAATGATAAATTTAATGAGTTTAAAGAGGCTTTTGACAGTATTATTAATCAAACATACTCT
AACTTTGTTTTCTTTATTTATGCCGATGGCAAGATAAATCCAGATATTGAAAGTTTTTTGCAAAATTATAATTCGCA
ACATGTGATTTTCATTAGAAGTGAAAAAAATAAGGGATTAGCTTATGCTTTGAATAAATTAATATCAGTGGCGCTAG
TTAATAATGAATTTGATTTTATTGCCAGAATGGATGCTGATGATATAAGTTTAGAAGATAGATTTCAACGACAAATA
GATTTTTTTATGGCTAACCCTGAGTTAGATGTTGTTGGTGGGTTGTGTCAAGAGTTTGGTGGTGAGTCAGCAAGAGA
AAAATGCAAGATCTATTTTACAGATGAAGCCATTAAAAACAATGCCTTTAAAAAATGTCCTTTCGTTCATCCAACCG
TGATGTTTAAATCGTCTGTATTTAATAACGGGATGCGATACCCTGAAAATCAGCCACTGACAGAAGATATGTGCTTA
TGGCTCGAGTTAATATCCGCGAATAAAAAGTTTGCAAATATTGACTCCGTTCTTCTAAAGTACAGAATTAATGAGCA
GACGCTAAAAAGAAGAAGAGGETTGAATAAGGCTGTAACCGAATTATCCACAAGATTGTATTACGCCAAGAAACTTA
AAATGCTTAGCGTACGGAATTTGTTATATATATTAATCCATTTTGGTGTCAGAGTCCTTCCTTCAAATATATTTAAA
CTTGTACATAAGTTAAATTAAAATGAATAAAACTGETTATACATATGATGTATGGGATACTATTTTAAAACGTCATT
GTTTACCATCATATACGTTAGAGGTTTCGTTGTACTGGCTACTTTTGTATCTTGGAAAACCTGAGAAACATAAGGAT
GTGTATAAAAAAGTAAAAGCACTGGAGAGTGATTTTTTTAATCGAAAAGAAGAATATTTTATTGAAGAGCTTATTGT
TGGGATATTAATTGAAAATAATTTAATGAATGGATTTTGCAATAACTCTGTGACTGCTGCTTGGGAACAAATATATT
ATTTCGTAGAGAGAAATTGTACATATAAAAATAAAGAGGTTATAGAGTTAATTGGGAGTGACTATGGGGACAAATTA
TTTATTTCTGATTTTTATACTGACTCCAGTTTCATAAAGAAATTGCTACACCACCACCAAGTATCGGTTTTAGATGG
TGTGACGTCAGCTGAGTATGGCGTCAGTAAGCATATGGGTTCATTGTATGAAAAAATAGAACTATTGAAGCAAAGGA
AATGGATCCATACTGGAGATAATGAATTAGCTGATATAAAAATGGCAAAGCGTGCAGGTGCCCAAGTTAGACTTATA
AAATCCAAAAAGAAGAAAATAAATAAGTTAAAAATAAAAAATGACTATGAGCGGTTAGCATTTATTATTTTGTCCTT
TAGCCTGTTTATATTAAAAACTTCCAGAAAGAAAAAGTGCAATAAGATCTACTTTTTTACAAGGGAGGGGGTTTTTT
TCAAAAAAAACTTTGATCTTCTACTAAACCAAATACCTAACATATTCCCATCTATAACTACGGAAATATTGCCAATC
AGTCGAGTAGCCAGCTTAGCATTGAAGTTTAATGAGTCTGATGAATTTTTAGGATTTAATGATGCATTAACCCAGTA
TGGCTATGGAGTGTCTACGTTTCTTAGTTTCTTTCATCTTGATGATTGTTACTCCGAGCTCACAACAAAGTATCATG
ATGTAAATGATTTATTATCTCACAGAAATGATCCTTTGGTAAAACAACTAATAAAAAGTATAGAGGATAAAAAAAAA
CGAACAGAAAATTTTCTTACATCCATTGAGTTCGATTCTGAGCCAAGTATCATCGTGGATATTGGTTGGCGGGGTAG
CATCCAAGATAATTTAATGTCGCGGAAAAATAATTATATTCAACATGGATGTTATCTCGGATTGTTTGATTTTTACC
CAGGCCAGTCAGGTCAGAATAAATCATCTGTAATTTTTGATAACAACAGATTTAGACAAAAATGGACCATGAAAGGT
GTAGCTTTTATGGAGACGGTTTTCAATGCTTTGGACGGAAGTGTTGTCGATTATGTAAACAATAAACCGAAAAGAAA
AGAAAATTACGCTGAGATAGAAAATTCAAAACATTTAATAGTTATGCAAGATGCTATCATTGAGGAATTTATGAATC
TATCTATTATGTTAAAAGAGGGAAAGTTAAATATCAATGATATTGAAAGATTAGCTATTGAAAGTTACAAAAAAATA
GTAACTAACCCATCTAAAGAAATAGCTGATTTTTATGTTGGCAGTATCCAAAATGAAAGCTTCGGTTTGAATGATTT
TATCGACAAAGAATTTAACATTAGCGCAGTAGATGTTTTCCGAAGTTTTTTTTCTAAAAGTAAAAGAAATGAAATAA
GAATAAAATTGCATAGAAATGGATGGCGTGAGTCGATACTTAAATCCTCTAAAGTATCGCTGAGCGCAAAATTATGT
TCACTTTTAATTAGATAAAATATGAATATTTCAGTAATAATTTGTTGCTATAACTCAGAAAATAGAATCGTAAACAC
CCTTTTTCATCTTTCACAATGTGTATCTCTATCCAAGGGGTTAATTGACGAGATTATTCTTGTTGACAATAACTCAT
CTGATGAAACTGTTAAAACAGCTGAGGATTTTTGGCGTAATACGGGGGAGGAGCAAATCTTTAAAATTGTTTTTGAA
TCGATTCCGGGCCTTGTTCATGCTAGACGGCGAGGGCTGGACGAGTCCAAAAATGAGATTGTTGTTTTTTGTGATGA
TGATAATTGGCTGGTACCATCTTATTTTACAAAACTAGAGAAATATTTTGAAAACAAACAAGTAGGCGCTGTCGGGG
GACGCGGTATTCCTATTGCTAATACCGTTATTCCTGAGTGGTTTTTTGAGAATCTGGATGCATATGCATGTTCTGCA
CAAACTTCTAACATGTTGTATGGTGCTAGCTTGGCTATTCGGTCTAGTATCTTAAAAAAATTTTATACGGATGCETT
ATCAGTAAAGCTAAAGGGTAGAACAAAAGACAGTCTGGATTCTGGGGAAGATAACTTATTGTGTAATTTTGTCAAAA
AACAAGGATACACTCTGATACCTGACAATACACCATTTTGCCATTTCATGGAAAGCAAGCGATTGGATGTCGATTAT
CTATATAAACTGGCTGAATCATTTGGAAAAGCAACGGCATCAATGAAAAACAATTCCTTCACCACCGATTATGTATA
TTTGACAGCAAAAATGTTATTAGCTATATCTAGGATCTTCTCTAAAAAAAATAAAAAGATATATTTAAAACGTGAGA
TTTCCTTTCTCAGAGGTTATTTGAGGTTTAGAAAGGAATCGAAGTAATGAGAGGGGGAATGCTTGTAATTGCGTGGT
ATTCTATTTTTCTGTTGATTTCAGCTACGGGCATGACCGGTCTAAATAAGCTGAGTACTGAAGCTATTCTGTGCTAT
ATTGCATTTTTATTCTGTTTTTTCTTTGGCATGATGTTAGGTGGTGTTTCGCTCCCAAACGGAATGAAAATGTTGCA
ACAGGAGCAAAAGATAAAGAAAAGGCATGCTTCTGCCATAGTTCTAATTTATTGCTTTTTTATTTTTTATTCTTTAA
TTGCTCTTTTAAATATATATTATTTCAATAAGGTTGATCTATCATCTTATAGACAAGCATTTTTTGATGGTTCAAGA
GATAATGTTAAATACTTCTATGGCGTGTTTGGCTTTTACTTCTATTATTTACTTAGCCTGTTAGTATTCGCCTTAGT
TCCTTATACTATTATAACGAACAGCAAAAAGACTATTTCATTATGTTTCTTTGCCTTGCTGCTATACGATATTATTT
TCCTTTCAAGAACTGGTGTTTATTACTTTATATTATCTTATATAGTATCGAGTATTCTTCTCAAGAAAGGAATCAAG
AAAATTATAATTGTAGGGATGGTTGCCTTATTATTCTCATTGATAATGAGTTACACAAGGGATATGAAGAGTGACAT
CATTGGTACATTATTTAGCTCAATTATAAATTATCATATAGCTCCGTTTGTCTTGTTTGATGAAAATATAATATCAA
ATCATTTGATTAAATATAATGGGGCTGGCTTGGCATCGTTGGGGATATATAATATCATTTTTTATCCCATTGATTCG
TCTATCATGTCATCAATAAACGATTTTAGAGCACAGTTGAATGAATTCTATGATTTGGGAGTAAATCGTTATTTGCC
ATATAATGCCTATTATACTTCTTTGGGTGGGGTATATATAGATAGCGGCCTGATTGGTAGCATGATGGTTAGTTTTA
TGACTGGTTTTATTATTGTCGGAATCGAGAAGAGTGCTTTTAAATCAACTAAGTTCTTAGTCTCTTCTGTGTTTTTT
ACTACATTATGTATGGAATCTATATTTTCCCCAATAACACTAAATATTTTTGCCTTTACAATAATTGTCTACTTAGT
TGTTTTGATGGTCCTTAAATATGAAAATAGCGATGATAATACCATCTTTAAAAAATACCGCGCCAAATAATATAGCC
CTTTCAATATGTGAAGGGGCAAGGAAGCATGGTGTCAATATAGATATTTATTATCTTGATGAAATTGTAGAGTTGGA
TATAGACGAGTCAGAACTGAATATAAATAGGCTAAATGATAGAACCATTTTAAACAGTTATGATATTGTTCACTCTC
ATGGCTTAAGACCCGATGTTATTAATTGTAAGACCAGCAAAAATGTCATTAATGTAAGTACGCTTCATAGTTTTATT
TTAGATGATCTTAAGAATAAATATGGAATAAAAGGATATCTGATCGCTTTTGCTTGGCTTAAATTATTAAAGCGATT
TGATTCATGTATTGCAATTAGTGAGGCGACTAAACGGTTTTATGTTAAACATGGTCTTAAATGCCACCATATATATA
ATGGAATCGACTTTCCTGACATCGAATTATATCAAGATAACTCAAGGCTAAATAACGGCAAAATAAATATAGTATCG
ATCGCGCATCTGGAAAAAATAAAAGGCTTAGAGCAACTCCTATATCTAGCCGCGGAGCGAGAAGAGTTCCATGTTCA
TATAATTGGTGAAGGAACTTACCGTGGGAAATTAACAAATATTATTGATAAGTTTGATTTATCTCAGCGTGTAACTT
TGCATGGATATATATCAAATGCGAGTTCTATGTTGGGGAAATTTGATGTTTATGTGCAACCATCTAAAAGTGAGGGG
TTTGGCATTGCAGTCATTGAAGCTCTTGTAAATAAAATCCCAACAGTTTGCTCAGACATTGAAGTGTTCAGAGAGTT
ATTTGGCTCAGGAGAAGTTGAATTTTTCAAGTTAGACGATATTAATTCACTTTACGATGCAATTTCAAGTGCATTAA
CCAAGACAGATATGTCTCGAGATGCAAGTGCCACAGCGATAAGCCAAAAATTTTCTTCAGAAGTGATGTCATTGAAC
TATTTAAGTTGGTATAAAAAATTATATGAAGAAAGAAATTTATAAATATGGCACAATAATATTATGCTATGGAATTA
ACTTTATACTACCTATTCTTTTCTTTAAAGAAATAAGTTTAATATTATCTCCAATAGTATTATCATCATTTTTCTTT
GTTTTGGCATTGCTAATGTATTGTCAATTCATTGTTGAATACGGGTTGCAAATATCATTATTAAGAAAGTTAAATGA
AAATAAGAGTGATTTAAGTCGGCTTTTATCTACTATTCTTGGACTAAAAGTAATACTATTCGTCCTATGCGCAGTAT
TCATTTATTGTATTTTGCTATATAATAATGAAGTTATATTTTTTATACTTGTTTTCATTCTGCTAGGAAACGTATTC
TCATGCCAGTTCCTCTATCAAGTAGTTGATCAGCTCCATTTTTTTTATGTATTAAATTCGCTTGTTAAGCTAATTTT
CATACCCCTCATTTTTATCAATGATAATTATATCTATCTGATGATATGTTATAGTTGTTTCAATATCCTACCTAACT
TAGCTGCACTTGCTTATTTTTTATATAAGAATAAAGTTAAGGTTGTAAAAGTTCCTGCTTGTGAAATTTATAATTTA
TCTAAGGAATGTTTTAGCTATTTTATCTCGAATATTTCGATAACATTATACACTAATTTTTATCAGATCTTATTAGG
CATTATATCGCCCGCTTATCTGGCCGCGTATGCATTAAGTGACAAAATTATTAGGGGGATCGTGTCAGGGAATTATG
TGTTTATACAGATAGTACAAGTACAATACTTAAATGATGAATCAAAGTTCATCAATGGTACATGGAAAACGATTGTT
TCATTATTATTACTGGGGGGGATGGAGTTTTTATTTATTTTCTTTGGAGCGGATTTTTTCTCTCGTTATTTATTCCC
TACAATATCTTTACTGCCAATTTTTCTAAAACTAATGTCATTGCTAATTGTTGTTATCTTGTTGAGTAACTTTTTTG
CAATGGTTTATCTTCCGTGTTCAGGCAATACTTTATTCCTCTCACGTGTTCTGATTTTTGTTTCTATTGTGTCCGTA
GTAGTTGCCCCGATACTAATATGGCTCTATGCAGGTTATGGTGCAGTCATTTCCGCTATATTTTCAGAGTTATTAGT
TCTGATTTTATGCTATGGTTTATATTTGAAATTAAAACACAAACTCTAAGGCGCCACGCGTCCTAAGGTTAATTAAG
CGGCCGCGTCGACTCGAGGATCCGTGTAGGCTGGAGCTGCTTCGAAGTTCCTATACTTTCTAGAGAATAGGAACTTC
GGAATAGGAACTAAGGAGGATATTCATATGGATAAAGCCGTAAGCATATAAGCATGGATAAGCTATTTATACTTTAA
TAAGTACTTTGTATACTTATTTGCGAACATTCCAGGCCGCGAGCATTCAGCGCGGTGATCACACCTGACAGGAGTAT
GTAATGTCCAAGCAACAGATCGGCGTAGTCGGTATGGCAGTGATGGGACGCAACCTTGCGCTCAACATCGAAAGCCG
TGGTTATACCGTCTCTATTTTCAACCGTTCCCGTGAGAAGACGGAAGAAGTGATTGCCGAAAATCCAGGCAAGAAAC
TGGTTCCTTACTATACGGTGAAAGAGTTTGTCGAATCTCTGGAAACGCCTCGTCGCATCCTGTTAATGGTGAAAGCA
GGTGCAGGCACGGATGCTGCTATTGATTCCCTCAAACCATATCTCGATAAAGGAGACATCATCATTGATGGTGGTAA
CACCTTCTTCCAGGACACTATTCGTCGTAATCGTGAGCTTTCAGCAGAGGGCTTTAACTTCATCGGTACCGGTGTTT
CTGGCGGTGAAGAGGGGGCGCTGAAAGGTCCTTCTATTATGCCTGGTGGCCAGAAAGAAGCCTATGAATTGGTAGCA
CCGATCCTGACCAAAATCGCCGCCGTAGCTGAAGACGGTGAACCATGCGTTACCTATATTGGTGCCGATGGCGCAGG
TCACTATGTGAAGATGGTTCACAACGGTATTGAATACGGCGATATGCAGCTGATTGCTGAAGCCTATTCTCTGCTTA
AAGGTGGCCTGAACCTCACCAACGAAGAACTGGCGCAGACCTTTACCGAGTGGAATAACGGTGAACTGAGCAGTTAC
CTGATCGACATCACCAAAGATATCTTCACCAAAAAAGATGAAGACGGTAACTACCTGGTTGATGTGATCCTGGATGA
AGCGGCTAACAAAGGTACCGGTAAATGGACCAGCCAGAGCGCGCTGGATCTCGGCGAACCGCTGTCGCTGATTACCG
AGTCTGTGTTTGCACGTTATATCTCTTCTCTGAAAGATCAGCGTGTTGCCGCATCTAAAGTTCTCTCTGGTCCGCAA
GCACAGCCAGCAGGCGACAAGGCTGAGTTCATCGAAAAAGTTCGTCGTGCGCTGTATCTGGGCAAAATCGTTTCTTA
CGCCCAGGGCTTCTCTCAGCTGCGTGCTGCGTCTGAAGAGTACAACTGGGATCTGAACTACGGCGAAATCGCGAAGA
TTTTCCGTGCTGGCTGCATCATCCGTGCGCAGTTCCTGCAGAAAATCACCGATGCTTATGCCGAAAATCCACAGATC
GCTAACCTGTTGCTGGCTCCGTACTTCAAGCAAATTGCCGATGACTACCAGCAGGCGCTGCGTGATGTCGTTGCTTA
TGCAGTACAGAACGGTATTCCGGTTCCGACCTTCTCCGCAGCGGTTGCCTATTACGACAGCTACCGTGCTGCTGTTC
TGCCTGCGAACCTGATCCAGGCACAGCGTGACTATTTTGGTGCGCATACTTATAAGCGTATTGATAAAGAAGGTGTG
TTCCATACCGAATGGCTGGATTAA
SEQ ID NO: 4 (Example PglB sequence (‘wild-type’)
MLKKEYLKNPYLVLFAMIILAYVFSVECRFYWVWWASEFNEYFFNNQLMIISNDGYAFAEGARDMIAGFHQPNDLSY
YGSSLSALTYWLYKITPFSFESIILYMSTFLSSLVVIPTILLANEYKRPLMGFVAALLASIANSYYNRIMSGYYDTD
MLVIVLPMFILFFMVRMILKKDFFSLIALPLFIGIYLWWYPSSYTLNVALIGLFLIYTLIFHRKEKIFYIAVILSSL
TLSNIAWFYQSAIIVILFALFALEQKRLNFMIIGILGSATLIFLILSGGVDPILYQLKFYIFRSDESANLTQGFMYF
NVNQTIQEVENVDLSEFMRRISGSEIVFLFSLFGFVWLLRKHKSMIMALPILVLGFLALKGGLRFTIYSVPVMALGF
GFLLSEFKAIMVKKYSQLTSNVCIVFATILTLAPVFIHIYNYKAPTVFSQNEASLLNQLKNIANREDYVVTWWDYGY
PVRYYSDVKTLVDGGKHLGKDNFFPSFALSKDEQAAANMARLSVEYTEKSFYAPQNDILKTDILQAMMKDYNQSNVD
LFLASLSKPDFKIDTPKTRDIYLYMPARMSLIFSTVASFSFINLDTGVLDKPFTFSTAYPLDVKNGEIYLSNGVVLS
DDFRSFKIGDNVVSVNSIVEINSIKQGEYKITPIDDKAQFYIFYLKDSAIPYAQFILMDKTMFNSAYVQMFFLGNYD
KNLFDLVINSRDAKVFKLKI
SEQ ID NO: 5 (Example CRM197 sequence)
GADDVVDSSKSFVMENFSSYHGTKPGYVDSIQKGIQKPKSGTQGNYDDDWKEFYSTDNKYDAAGYSVDNENPLSGKA
GGVVKVTYPGLTKVLALKVDNAETIKKELGLSLTEPLMEQVGTEEFIKRFGDGASRVVLSLPFAEGSSSVEYINNWE
QAKALSVELEINFETRGKRGQDAMYEYMAQACAGNRVRRSVGSSLSCINLDWDVIRDKTKTKIESLKEHGPIKNKMS
ESPNKTVSEEKAKQYLEEFHQTALEHPELSELKTVTGTNPVFAGANYAAWAVNVAQVIDSETADNLEKTTAALSILP
GIGSVMGIADGAVHHNTEEIVAQSIALSSLMVAQAIPLVGELVDIGFAAYNFVESIINLFQVVHNSYNRPAYSPGHK
TQPFLHDGYAVSWNTVEDSIIRTGFQGESGHDIKITAENTPLPIAGVLLPTIPGKLDVNKSKTHISVNGRKIRMRCR
AIDGDVTFCRPKSPVYVGNGVHANLHVAFHRSSSEKIHSNEISSDSIGVLGYQKTVDHTKVNSKLSLFFEIKS
SEQ ID NO: 6 rfb cluster E. coli O21
ATGACGAATTTAAAAGCAGTTATTCCTGTAGCGGGTCTCGGGATGCATATGTTGCCTGCCACTAAGGCGATACCCAA
AGAGATGCTACCAATCGTCGACAAGCCAATGATTCAGTACATTGTTGACGAGATTGTGGCTGCAGGGATCAAAGAAA
TCCTCCTGGTAACTCACGCGTCCAAGAACGCGGTCGAAAACCACTTCGACACCTCTTATGAGTTAGAATCACTCCTT
GAGCAGCGCGTGAAGCGTCAACTGCTGGCGGAAGTACAGTCCATCTGTCCGCCGGGCGTGACCATTATGAACGTGCG
TCAGGGCGAACCTTTAGGTTTAGGCCACTCCATTTTGTGTGCGCGACCTGCCATTGGTGACAACCCATTTGTCGTGG
TACTGCCAGACGTTGTGATCGACGATGCCAGCGCCGACCCGCTACGTTACAACCTTGCTGCCATGATTGCACGTTTC
AACGAAACGGGCCGCAGCCAGGTGCTGGCAAAACGTATGCCGGGTGACCTCTCTGAATACTCCGTCATCCAGACTAA
AGAGCCGCTGGACCGTGAGGGTAAAGTCAGCCGCATTGTTGAATTTATCGAAAAACCGGATCAGCCGCAGACGCTGG
ACTCAGACATCATGGCCGTAGGTCGCTATGTGCTTTCTGCCGATATTTGGCCGGAACTGGAACGTACTCAGCCTGGT
GCATGGGGACGTATTCAGCTGACTGATGCTATTGCCGAGCTGGCGAAAAAACAATCCGTTGATGCAATGCTGATGAC
CGGCGACAGTTACGACTGCGGCAAAAAAATGGGCTATATGCAGGCGTTTGTGAAGTATGGCCTACGCAACCTGAAAG
AAGGGGCGAAGTTCCGTAAAGGTATTGAGAAGCTGTTAAGCGAATAATGAAAATCTGACCGGATGTAACGGTTGATA
AGAAAATTATAACGGCAGTGAAAATTCGCAGCAAAAGTAATTTGTTGCGAATCTTCCTGCCGTTGTTTTATATAAAC
CATCAGAATAACAACGAGTTAGCAGTAGGGTTTTATTCAAAGTTTTCCAGGATTTTCCTTGTTTCCAGAGCGGATTG
GTAAGACAATTAGCGTTTGAATTTTTCGGGTTTAGCGCGAGTGGGTAACGCTCGTCACATCATAGGCATGCATGCAG
TGCTCTGGTAGCTGTAAAGCCAGGGGCGGTAGCGTGCATTAATACCTCTATTAATCAAACTGAGAGCCGCTTATTTC
ACAGCATGCTCTGAAGTAATATGGAATAAATTAAGCTAGAATGTTTACAATTAAAACTGTATTTGTGTTGAGGATAT
TCGGCGCTATACTTGCGCTGTCAACGAGTGTGATGATATCAAGAACGCTCAATATGAGTGAGGCTGGTGGGGTTTTC
TTCCTATTATCAGTAACTGGAATAATATCGTCCCTATCCTCTTTAGGACAAAATAATTTAATTTTGAAAAAATGTGC
CTCAGTTAAATATGCATTAAAAGCACGAGGCGTTTTTTTTCTTAGATGCATAAAACGGAGTGTTATATCCAGTATTA
TCTTATCTATCATTTTAATACCTATAATTCATTATTTTTCGCCACAATTATTATTACGAAATACATGGTGTTTGGCG
ATTCTGTTGATAATATCATCGTCAGTAAATATATTGTTATATTCCTTTTTACAGTCTCAAGGTCTTGTCACTTTTAG
TGTTGTTCTGCAGTATATATTTCAACCCTTATTGTTTATTTTTTTTGTATTACTTATTGTTCTGATTAAAAATGAAA
GTGTTTTACTGGTGTCTTTATCATACTTCTTCTCAATTATTCTTATTGGAGGAGGGGGGGGGGTATACTCGTATTAT
AAATTTAGGGACGATTTCAATCAGTTAGAAACAGAGTCAATACCGTTTAGACTGAAAGAATTATCAGAGTATTTCAT
TGGTAACTCATTAGGAATGCTGATTGTTCAATCTTATGTTGTACTTTCCGGTTTGTTGCTCCCAGCTGCTGATGTCG
CTATTATTGCTGTGTCTGATAGAATATCCTTGGTTATAAATTTATTTGCAATGTCAATTAGCACAATTCTGGCACCT
AAGGTCGCCAGTTTATACAGCCAAAGTAAAATGAATGAAATAAAAGAGCTAACTAAAAAAGCTATGTTCTTCATAAT
GATACCATGTGTTTTAATGGCTCTTCTGTTTCCCTTTTTTTCAGGGCTGATATTATCTATATTTGGTGTTCAATACA
GTAATGCTAGCGAAGTGTTAATTATTTTAGTTATGACGCAGATTATTAATGCAATATTTTGCCCTGTATATGTTTTT
TTAAATATGAGTGATCGGCAAGGTTTTATAAGTAAATTGCATATATATATGCTTATACCGTCTCTTGTTATAACATT
TTATCTGACTAACATATTTGGTGTTGTTGGAATAGCTGTGTCTAAGTTAATTGTCGTGTCATTGATTAATATAATCC
CATTGATTTACTGTATTTTATTGTCTAATCGAGTGATAAAGTGATGAGTTTATATTCTATTTTAAAAAAGTTAATTT
TATCTTTTCATTACTATCTTTTTACTATTTCCTTTTTATCTGCGAAAAACGATAAGAAGTACTCCAATCGGTTAGAT
TTTGATGATTATGTTGCAGCTAAGCTAAGAAAACAGAATGAATATAAGAGGAAAATACCAAGTGTCTCTGTTATATA
TAGAGTCAAAAACGGATCTGAATATATAGAGGCATCTATATTGTCTATTTCGGGGTTAGCATCACAAATAATTGTTG
TAGATAATAATTCTACAGATAATACAAGAGATATTGTAGAAAGATTATCGAATCAATTAGCTGATGTTTGCTCTATA
CAACTTTACTCTTATGATAAAAATCTCGCTATTGCTGGAGAAGGGTATAAAGATCAGGTGACAAAGGGAGATGGTTC
ACTGGCCGAATTTTATAATTTTAGTTTTTCTTTAGGTGATTGTGATTATTTAATGAAAGTCGATGCCCATTATATTT
TTTCGGCATACGGTGTTGATATTTTGCAAAAGCAGATAGCAAAAAATTATGATGGTGTCGTTTTTCGAGGGTTAGAG
TTTTTTGGGAAGTGGATGAGCAATGAGCTGTTTTTATATAAACGTTCACTGGGTCTGAAATATTGTGATGGCGAATT
ATATGAACAGTTAATGTGGGATAAATCAACCGTCAGGGTAAAAACTATCATTAAACCCCETTACTTGCATGTTAAAC
GGTTATCCTATGTTAAAAACCETTATAACACAAAGAAAGCCATATACGTAAAATACAATAAATAGGCAATAGAATGA
ATAATAAAAGAATATCAATAATAATACCAACTTATAATTCGAGCATAATATTAGAGGAAAATATTAAAATAATCAGT
AGCTATAAGCAGTGTAAGATTTTTGTAATTGATGATGGTTCAGTTTCTGATGAAGCGCGCAAAAATAAAATTATTTG
TAATAATTACGGGTGTAATTACACCTATACTCTTAATCAAGGCCCTTCTCACGCAAGATATGTAGGCTTATTGGATT
CAGATACAGAATTTTGTTTTTTTCTTGATACTGATGATTATATTGCTGAATTTGCTCTTGATTGGGGTGTTAATTAT
TTATTAGAAAATAATGAGGTTGATGCCATCGTCTTTAGAAGTGATTATGTGGAAGAGTTTAATTGTAAAAACGAGAC
AGGTAATATTTTCACAATAAAAAAATATAACAGAGGATTAATACAAGAATGTATAGAGTGTTTAGGGTATCCAAAGC
AATTTACTCTTGGTTGGAATCAATCTAATACGTTGTATCGTAGAAATAAAATCATTGATGCTTATAAAATAAGATAT
TTGACTTGGGGGGAAGATATACCATTAAAACTAAAACTAATTTCTGTTATGTCGCTAATTTCTGTGAGAACTCTGGG
AGGGTCTCAAATTAAAATATCCTATGGACGGGGATATAAATATACACCAAAACAAATAGTTGAGCTTGCTCTGGAGG
TTTACAGAACGTCCCTAAATAATGCATTTTGTCTTGCATTCATAACAGTTATAAGATATATGCTTTCATATTTTTAT
AAAAGATTAAAACAACTGTCCAGTAAAAGGAAGTGAGAATGAAAAGAAAACTTGTTGACTTTTGTATAATATCATTA
CCTCAACATAATGAAAGGAGAGATAAATTAAAAAATGAAATGGCGAAGTATGACATAGAATGTCGTGTTTCTCATGC
TATTGATGGACGGAAATTATTGGCAGAAAAATACTTTTCTTTATTTAAAATTAGGTCCTCAAAGATGTTTGGAAGAG
GTTTTTTAACTCCGTCGGAATTGGGATGTTTCTTGAGTCATAAAAAAGCTTTAACGGAATTTTTGGCGAGTGGAAGG
AAATGGTTGGTGGTTTTGGAAGATGACGTTTTGCCCAAGGAAAATGTTAAATATTTGGACGAAATGATTAATTCATT
CTGCTCATCAAGTGTCTATATTTTGGGTGGACAGGATGGTCTAAAAAGCTTTAGTCGTGTCATAATGGGGAGAAAAT
CAATATGTGGGGTGCGAAAAGTAATATTAGGTACACATCGTTGGTTATATAGGACATGCTGTTATTGTGTGGATATT
AAAGGCGCTGAGAGAATTCTGAGGTTAATGGAAGAAAATAGTTTCTTTTGTGATGACTGGAGTTACATTGTAAGAAA
TGCGAAACTGGATAATGTATTTTATGGTCAATACTTTTCTCATCCTGTAAATTTAAACTCTAGTTCTATCGAAGCTG
AACGTCTTTTTATTGCAGAAAAATGATATATATTATAATTAATACTATTGTGCTTTTTTATATGTTTTTGTTTAATG
CATATAATCGTATTCCTTATGGGAAATTAATTATCGGGGGCATATGCGTTTTTTTTATGACTCTGTTGCCAGCATTT
CAATACGGTGTTGGAACAGATTATTTTAGCTATCAAAATATATATAATAATGCTAATGAACTCGATACCTTTTATCA
AAATAAGGAATATCTCTTTTATGGATATATGAGGCTATATCAACTGACTGGATGGGGATTCAAAGGTTTTATTGCGT
TAACTGCTTTATTACAATCTTTATTGGTGTTTGTTATTGTTTTTCAGTTATGGAAAAACTATGGTTATTCTCTTGTT
TTGGTTTTTTTTCTTTTTTGGGTTGTTACTAATTTATTTCATACCCAGATGAATATAATAAGAGCATCATTTTCAAT
TTATTTGTTTGCAATATCAATTCTCTATAAGTTTAGAGGGAAATTATTGTTATCTTTCATATTAATGGTTGCTGCTT
TAGGTTTTCATCGCTCTGCTTTGATTGGTTTCTGTTTTCTCGTAATTCCAGATAAGATGTATTTTTTTGCGTGTAAA
CATGCTTTTAAAATTTATGTGTTGACTTTTTTATTGTTTCTTTTCTCTTATCTTCAGCAAATTATTTATTATATAGT
GCAAAATTTATTCCCCTATTACTCACATTATTTATCCTCATTCGATGAAAGTTCTGTGAGTATAATGAATGTACTAA
CTAAAATGTATTGGATTCCATTTAATTTGCTATTCCTTATCTTATTAAGACTAAAGGTTTTTGTTATAAAAGATAAT
GAGCGAAAGTTAATTGGTATGTGGGCTTTAACGGTGAATGTTTATTTGTTAATGCTCAGTTTTGATTTTATTTCAAG
GGTTAACTACTATTTTGTGATATTTTATATAATCCCGATAATTTATGTTATTAAATACACTGTTAAGAATAGATACT
TCGTTTGTTTGTATCTCTCATTATTATACTGTTTTATTCCGTATCTATTAAAGGTTATTCTGTTTCCTATCGCAGAA
TTTTATTATAAATCATACTTGTTCTAATTTTGAATAATGATGGGGCTTTTTATGGGTAATGAAACAGTATCAATAAT
TATGCCAGCTTATAATGCTGAAGAAACTATTAAAGATAGTATTTTGTCAATTTTAAAACAAACGTATGAGGATTTTA
AATTATATATAATCAATGATAATTCGTCGGATTCAACAGAACATATTATTAAGTCTATTATCGATGAACGTATTGTT
TATTTACTAAATCGAAATGGTAAGGGAGTATCTTCGGCTAGAAATGTTGGGATTGCTGCTTGTAATGGTAGATATAT
TGCATTTTGTGATAGTGATGATGTTTGGTTTGAGACTAAACTCGAAGAACAACTGAAAATATTGAGTGCTGGAAATT
ATAAAGTTGTTTGTTCAAACTATGAGGTTTTTTATGCTGATACAAATGTAATAAAAGAAAGAAGGTTTAAGGAAGTT
ATTACATATAATAATATGCTGCAATCTAATCATATTGGTAACTTAACTGGTATTTATGACTCAACTCAGATAGGGAA
AGTATATCAACAGGAAATTGGTCATGAAGATTACCTAATGTGGTTAACAATAGTAAAAAAAGCAAAGCTGGTTTATT
GCATACAGAAAAATCTTGCCAGATATTATATACATAATACGGGGTTGTCCTCAAATAAGTTCACTGCAGCTATGTGG
CAATGGAATATTTATAGAAGAGTATTGTCTTTTTCATTATTCAAGTCATTAGTTCTTTTTTTTATTTATTCAGTAAG
AGCTCTTGCTAAGAGACTTTAAAGATCTAAGCTTAGGAGGTTTCACCTATGAACGATAACGTTTTGCTCATAGGAGC
TTCCGGATTCGTAGGAACCCGACTACTTGAAACGGCAATTGCTGACTTTAATATCAAGAACCTGGACAAACAGCAGA
GCCACTTTTATCCAGAAATCACACAGATTGGCGATGTTCGTGATCAACAGGCACTCGACCAGGCGTTAGCCGGTTTT
GACACTGTTGTGCTACTGGCAGCGGAACACCGCGATGACGTCAGCCCTACTTCTCTCTATTATGATGTCAATGTTCA
GGGTACCCGCAATGTGCTGGCGGCCATGGAAAAAAATGGTGTTAAAAATATCATCTTTACCAGTTCCGTTGCTGETT
ATGGTTTGAACAAACACAACCCTGACGAAAACCATCCACACGACCCTTTCAACCACTACGGCAAAAGCAAGTGGCAG
GCGGAGGAAGTGCTGCGTGAATGGTATAACAAAGCACCAACAGAACGTTCATTAACTATCATCCGTCCTACCGTTAT
CTTCGGTGAACGCAACCGCGGTAACGTCTATAACTTGCTGAAACAGATCGCTGGCGGCAAGTTTATGATGGTGGGCG
CAGGGACTAACTATAAGTCCATGGCTTATGTTGGAAACATTGTTGAGTTTATCAAGTACAAACTGAAGAATGTTGCC
GCAGGTTATGAGGTTTATAACTACGTTGATAAGCCAGACCTGAACATGAACCAGTTGGTTGCTGAAGTTGAACAAAG
CCTGAACAAAAAGATCCCTTCTATGCACTTGCCTTACCCACTAGGAATGCTGGGTGGATATTGTTTTGATATCCTGA
GCAAAATTACGGGCAAAAAATACGCTGTCAGCTCAGTGCGCGTGAAAAAATTCTGCGCAACAACACAGTTTGACGCA
ACGAAAGTGCATTCTTCAGGTTTTGTGGCACCGTATACGCTGTCGCAAGGTCTGGATCGAACTCTGCAGTATGAATT
CGTTCATGCCAAAAAAGACGACATAACGTTTGTTTCTGAGTAAACGCGTTTAATTAAGCGATCGCACTAGTGGATCC
GCGGCCGCGAGCTCGTCGACTCGAGGATCCGTGTAGGCTGGAGCTGCTTCGAAGTTCCTATACTTTCTAGAGAATAG
GAACTTCGGAATAGGAACTAAGGAGGATATTCATATGGATAAAGCCGTAAGCATATAAGCATGGATAAGCTATTTAT
ACTTTAATAAGTACTTTGTATACTTATTTGCGAACATTCCAGGCCGCGAGCATTCAGCGCGGTGATCACACCTGACA
GGAGTATGTAATGTCCAAGCAACAGATCGGCGTAGTCGGTATGGCAGTGATGGGACGCAACCTTGCGCTCAACATCG
AAAGCCGTGGTTATACCGTCTCTATTTTCAACCGTTCCCGTGAGAAGACGGAAGAAGTGATTGCCGAAAATCCAGGC
AAGAAACTGGTTCCTTACTATACGGTGAAAGAGTTTGTCGAATCTCTGGAAACGCCTCGTCGCATCCTGTTAATGGT
GAAAGCAGGTGCAGGCACGGATGCTGCTATTGATTCCCTCAAACCATATCTCGATAAAGGAGACATCATCATTGATG
GTGGTAACACCTTCTTCCAGGACACTATTCGTCGTAATCGTGAGCTTTCAGCAGAGGGCTTTAACTTCATCGGTACC
GGTGTTTCTGGCGGTGAAGAGGGGGCGCTGAAAGGTCCTTCTATTATGCCTGGTGGCCAGAAAGAAGCCTATGAATT
GGTAGCACCGATCCTGACCAAAATCGCCGCCGTAGCTGAAGACGGTGAACCATGCGTTACCTATATTGGTGCCGATG
GCGCAGGTCACTATGTGAAGATGGTTCACAACGGTATTGAATACGGCGATATGCAGCTGATTGCTGAAGCCTATTCT
CTGCTTAAAGGTGGCCTGAACCTCACCAACGAAGAACTGGCGCAGACCTTTACCGAGTGGAATAACGGTGAACTGAG
CAGTTACCTGATCGACATCACCAAAGATATCTTCACCAAAAAAGATGAAGACGGTAACTACCTGGTTGATGTGATCC
TGGATGAAGCGGCTAACAAAGGTACCGGTAAATGGACCAGCCAGAGCGCGCTGGATCTCGGCGAACCGCTGTCGCTG
ATTACCGAGTCTGTGTTTGCACGTTATATCTCTTCTCTGAAAGATCAGCGTGTTGCCGCATCTAAAGTTCTCTCTGG
TCCGCAAGCACAGCCAGCAGGCGACAAGGCTGAGTTCATCGAAAAAGTTCGTCGTGCGCTGTATCTGGGCAAAATCG
TTTCTTACGCCCAGGGCTTCTCTCAGCTGCGTGCTGCGTCTGAAGAGTACAACTGGGATCTGAACTACGGCGAAATC
GCGAAGATTTTCCGTGCTGGCTGCATCATCCGTGCGCAGTTCCTGCAGAAAATCACCGATGCTTATGCCGAAAATCC
ACAGATCGCTAACCTGTTGCTGGCTCCGTACTTCAAGCAAATTGCCGATGACTACCAGCAGGCGCTGCGTGATGTCG
TTGCTTATGCAGTACAGAACGGTATTCCGGTTCCGACCTTCTCCGCAGCGGTTGCCTATTACGACAGCTACCGTGCT
GCTGTTCTGCCTGCGAACCTGATCCAGGCACAGCGTGACTATTTTGGTGCGCATACTTATAAGCGTATTGATAAAGA
AGGTGTGTTCCATACCGAATGGCTGGATTAA
SEQ ID NO: 7 (nucleotide sequence encoding UDP-glucose 4-epimerase)
ATGAACGATAACGTTTTGCTCATAGGAGCTTCCGGATTCGTAGGAACCCGACTACTTGAAACGGCAATTGCTGACTT
TAATATCAAGAACCTGGACAAACAGCAGAGCCACTTTTATCCAGAAATCACACAGATTGGCGATGTTCGTGATCAAC
AGGCACTCGACCAGGCGTTAGCCGGTTTTGACACTGTTGTGCTACTGGCAGCGGAACACCGCGATGACGTCAGCCCT
ACTTCTCTCTATTATGATGTCAATGTTCAGGGTACCCGCAATGTGCTGGCGGCCATGGAAAAAAATGGTGTTAAAAA
TATCATCTTTACCAGTTCCGTTGCTGTTTATGGTTTGAACAAACACAACCCTGACGAAAACCATCCACACGACCCTT
TCAACCACTACGGCAAAAGCAAGTGGCAGGCGGAGGAAGTGCTGCGTGAATGGTATAACAAAGCACCAACAGAACGT
TCATTAACTATCATCCGTCCTACCGTTATCTTCGGEGAACGCAACCGCGGTAACGTCTATAACTTGCTGAAACAGAT
CGCTGGCGGCAAGTTTATGATGGTGGGCGCAGGGACTAACTATAAGTCCATGGCTTATGTTGGAAACATTGTTGAGT
TTATCAAGTACAAACTGAAGAATGTTGCCGCAGGTTATGAGGTTTATAACTACGTTGATAAGCCAGACCTGAACATG
AACCAGTTGGTTGCTGAAGTTGAACAAAGCCTGAACAAAAAGATCCCTTCTATGCACTTGCCTTACCCACTAGGAAT
GCTGGGTGGATATTGTTTTGATATCCTGAGCAAAATTACGGGCAAAAAATACGCTGTCAGCTCAGTGCGCGTGAAAA
AATTCTGCGCAACAACACAGTTTGACGCAACGAAAGTGCATTCTTCAGGTTTTGTGGCACCGTATACGCTGTCGCAA
GGTCTGGATCGAACTCTGCAGTATGAATTCGTTCATGCCAAAAAAGACGACATAACGTTTGTTTCTGAGTAA
SEQ ID NO: 8 (amino acid sequence UDP-glucose 4-epimerase)
MNDNVLLIGASGFVGTRLLETAIADFNIKNLDKQQSHFYPEITQIGDVRDQQALDQALAGFDTVVLLAAEHRDDVSP
TSLYYDVNVQGTRNVLAAMEKNGVKNIIFTSSVAVYGLNKHNPDENHPHDPFNHYGKSKWQAEEVLREWYNKAPTER
SLTIIRPTVIFGERNRGNVYNLLKQIAGGKFMMVGAGTNYKSMAYVGNIVEFIKYKLKNVAAGYEVYNYVDKPDLNM
NQLVAEVEQSLNKKIPSMHLPYPLGMLGGYCFDILSKITGKKYAVSSVRVKKFCATTQFDATKVHSSGFVAPYTLSQ
GLDRTLQYEFVHAKKDDITFVSE

1. A composition comprising E. coli O1, O2, O4, O6, O8, O15, O16, O18, O25, and O75 antigen polysaccharides and wherein the composition further comprises O21 antigen polysaccharide or O153 antigen polysaccharide or O21 and O153 antigen polysaccharides, wherein each of the antigen polysaccharides is independently covalently linked to a carrier protein.

2. The composition according to claim 1, wherein the O1 antigen is O1A, the O4 antigen is glucosylated (O4A), the O6 antigen is O6A, the O18 antigen is O18A, and the O25 antigen is O25B.

3. The composition according to claim 1, wherein
(i) the E. coli O1 antigen polysaccharide comprises the structure of Formula (O1A): 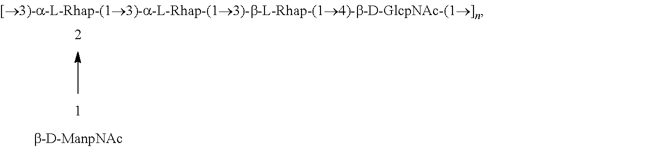
(ii) the E. coli O2 antigen polysaccharide comprises the structure of Formula (O2): 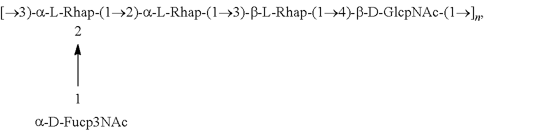
(iii) the E. coli O4 antigen polysaccharide comprises the structure of Formula (O4A): 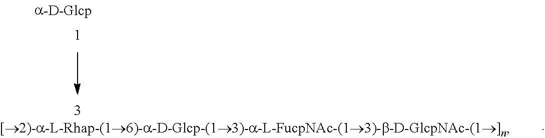
(iv) the E. coli O6 antigen polysaccharide comprises the structure of Formula (O6A): 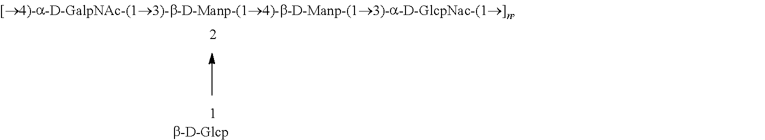
(v) the E. coli O8 antigen polysaccharide comprises the structure of Formula (O8): 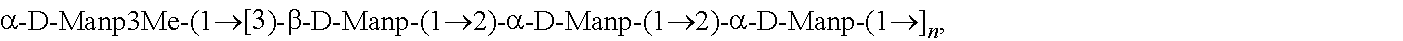
(vi) the E. coli O15 antigen polysaccharide comprises the structure of Formula (O15): 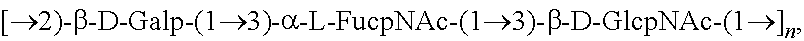
(vii) the E. coli O16 antigen polysaccharide comprises the structure of Formula (O16): 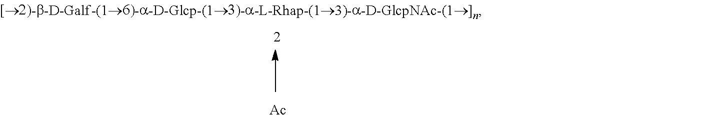
(viii) the E. coli O18 antigen polysaccharide comprises the structure of Formula (O18A): 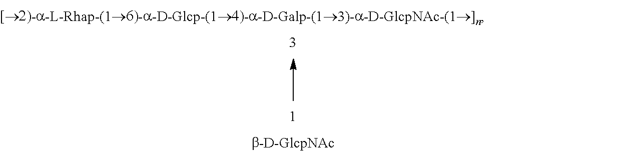
(ix) the E. coli O25 antigen polysaccharide comprises the structure of Formula (O25B): 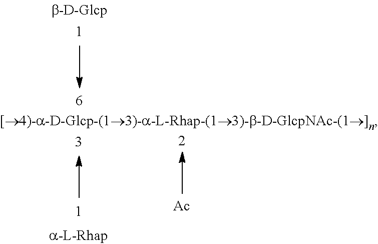
(x) the E. coli O75 antigen polysaccharide comprises the structure of Formula (O75): 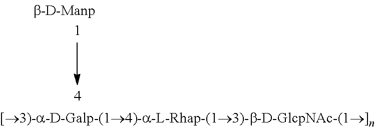
(xi) the E. coli O153 antigen polysaccharide comprises the structure of Formula (O153): 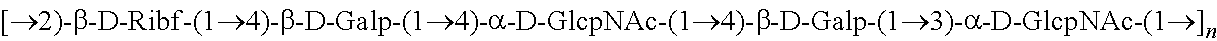
and,
(xii) the E. coli O21 antigen polysaccharide comprises the structure of Formula (O21): 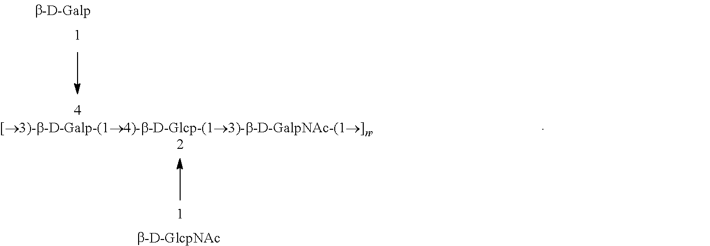
wherein each n is independently an integer of 1 to 40.

4. The composition according to claim 3, further comprising at least one additional E. coli antigen polysaccharide covalently linked to a carrier protein.

5. The composition according claim 1, wherein the E. coli O antigen polysaccharides present in the composition consist of:
(i) O1, O2, O4, O6, O8, O15, O16, O18, O25, O75 and O153;
(ii) O1, O2, O4, O6, O8, O15, O16, O18, O25, O75 and O21; or
(iii) O1, O2, O4, O6, O8, O15, O16, O18, O25, O75, O153 and O21.

6. The composition according to claim 3, wherein the carrier protein is detoxified exotoxin A of Pseudomonas aeruginosa (EPA) or CRM₁₉₇.

7. The composition claim 1, wherein the carrier protein is EPA.

8. The composition according to claim 6, wherein the carrier protein is EPA and comprises 1 to 20 glycosylation consensus sequences having the amino acid sequence Asn-X-Ser(Thr) wherein X can be any amino acid except Pro, and the E. coli antigen polysaccharides are covalently linked to the Asn residue.

9. The composition according to claim 3, wherein each carrier protein comprises the amino acid sequence of SEQ ID NO: 2, and the E. coli antigen polysaccharides are covalently linked to the Asn residue in a glycosylation site.

10. (canceled)

11. The composition according to claim 1, wherein the E. coli antigen polysaccharides are covalently linked to the carrier protein by bioconjugation.

12. The composition according to claim 11, wherein the E. coli antigen polysaccharides are covalently linked to an Asn residue in a glycosylation site in the carrier protein.

13. A pharmaceutical composition comprising a pharmaceutically acceptable excipient and the composition according to claim 1.

14. A method of inducing an immune response to an extra-intestinal pathogenic E. coli (ExPEC), in a subject, comprising administering to the subject the composition according to claim 1.

15. The method according to claim 14, wherein the immune response limits the severity of or prevents an invasive ExPEC disease in the subject.

16. A recombinant prokaryotic host cell for preparing a bioconjugate of an E. coli O153 antigen polysaccharide covalently linked to a carrier protein, the recombinant prokaryotic host cell comprising:
a. a nucleotide sequence of an rfb gene cluster for the O153 antigen polysaccharide;
b. a nucleotide sequence encoding the carrier protein comprising at least one glycosylation site comprising a glycosylation consensus sequence having sequence Asn-X-Ser(Thr) wherein X can be any amino acid except Pro; and
c. a nucleotide sequence encoding an oligosaccharyl transferase PglB,
wherein the E. coli O153 antigen polysaccharide comprises the structure of Formula (O153):
[→2)-β-D-Ribf-(1→4)-β-D-Galp-(1→4)-α-D-GlcpNAc-(1→4)-β-D-Galp-(1→3)-α-D-GlcpNAc-(1→]ₙ
wherein each n is independently an integer of 1 to 40.

17. A recombinant prokaryotic host cell for preparing a bioconjugate of an E. coli O21 antigen polysaccharide covalently linked to a carrier protein, the recombinant prokaryotic host cell comprising:
a. a nucleotide sequence of an rfb gene cluster for the O21 antigen polysaccharide;
b. a nucleotide sequence encoding the carrier protein comprising at least one glycosylation site comprising a glycosylation consensus sequence having sequence Asn-X-Ser(Thr) wherein X can be any amino acid except Pro; and
c. a nucleotide sequence encoding an oligosaccharyl transferase PglB;
d. a nucleotide sequence encoding an UDP-glucose 4-epimerase,
wherein the E. coli O21 antigen polysaccharide comprises the structure of Formula (O21): 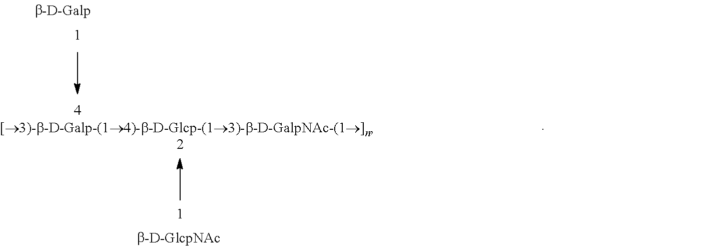
wherein each n is independently an integer of 1 to 40.

18. The recombinant prokaryotic host cell of claim 16, wherein the PglB comprises the amino acid mutations N311V, K482R, D483H, and A669V relative to wild-type PglB having the amino acid sequence of SEQ ID NO: 4.

19. The recombinant prokaryotic host cell of claim 17, wherein the PglB comprises the amino acid mutation N311V relative to wild-type PglB having the amino acid sequence of SEQ ID NO: 4.

20. A method of preparing a bioconjugate of an E. coli O153 antigen polysaccharide covalently linked to a carrier protein, the method comprising culturing the recombinant prokaryotic host cell of claim 18 to produce the bioconjugate.

21. A method of preparing a bioconjugate of an E. coli O21 antigen polysaccharide covalently linked to a carrier protein, the method comprising culturing the recombinant prokaryotic host cell of claim 19 to produce the bioconjugate.

22-35. (canceled)


SEQUENCE LISTING (8 sequences)

SEQ ID NO: 1 - skipped

SEQ ID NO: 2 - protein, 652 aa, synthetic construct
  source 1..652: note = EPA carrier protein comprising 4 glycosylation consensus sequences (EPA-4
GSGGGDQNATGSGGGKLAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKDNNNSTPTVISHRLHFPEGGSLAALTAHQACHLPLEAFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAKDQNRTKGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLAAPEAAGEVERLIGHPLPLRLDAITGPEEEGGRVTILGWPLAERTVVIPSAIPTDPRNVGGDLDPSSIPDKEQAISALPDYASQPGKPPREDLKLGSGGGDQNAT

SEQ ID NO: 3 - DNA, 9726 bp, synthetic construct
  source 1..9726: mol_type = other DNA; note = Rfb cluster O153
atgacgaatttaaaagcagttattcctgtagcgggtctcgggatgcatatgttgcctgccactaaggcgatacccaaagagatgctaccaatcgtcgacaagccaatgattcagtacattgttgacgagattgtggctgcagggatcaaagaaatcctcctggtaactcacgcgtccaagaacgcggtcgaaaaccacttcgacacctcttatgagttagaatcactccttgagcagcgcgtgaagcgtcaactgctggcggaagtacagtccatctgtccgccgggcgtgaccattatgaacgtgcgtcagggcgaacctttaggtttaggccactccattttgtgtgcgcgacctgccattggtgacaacccatttgtcgtggtactgccagacgttgtgatcgacgatgccagcgccgacccgctacgttacaaccttgctgccatgattgcacgtttcaacgaaacgggccgcagccaggtgctggcaaaacgtatgccgggtgacctctctgaatactccgtcatccagactaaagagccgctggaccgtgagggtaaagtcagccgcattgttgaatttatcgaaaaaccggatcagccgcagacgctggactcagacatcatggccgtaggtcgctatgtgctttctgccgatatttggccggaactggaacgtactcagcctggtgcatggggacgtattcagctgactgatgctattgccgagctggcgaaaaaacaatccgttgatgcaatgctgatgaccggcgacagttacgactgcggcaaaaaaatgggctatatgcaggcgtttgtgaagtatggcctacgcaacctgaaagaaggggcgaagttccgtaaaggtattgagaagctgttaagcgaataatgaaaatctgaccggatgtaacggttgataagaaaattataacggcagtgaaaattcgcagcaaaagtaatttgttgcgaatcttcctgccgttgttttatataaaccatcagaataacaacgagttagcagtagggttttattcaaagttttccaggattttccttgtttccagagcggattggtaagacaattagcgtttgaatttttcgggtttagcgcgagtgggtaacgctcgtcacatcataggcatgcatgcagtgctctggtagctgtaaagccaggggcggtagcgtgcattaatacctctattaatcaaactgagagccgcttatttcacagcatgctctgaagtaatatggaataaattaagctagcgatcgcatgaaaaataagtgcccacgaattgcagtaataatgtcggtttatattaatgataaatttaatgagtttaaagaggcttttgacagtattattaatcaaacatactctaactttgttttctttatttatgccgatggcaagataaatccagatattgaaagttttttgcaaaattataattcgcaacatgtgattttcattagaagtgaaaaaaataagggattagcttatgctttgaataaattaatatcagtggcgctagttaataatgaatttgattttattgccagaatggatgctgatgatataagtttagaagatagatttcaacgacaaatagatttttttatggctaaccctgagttagatgttgttggtgggttgtgtcaagagtttggtggtgagtcagcaagagaaaaatgcaagatctattttacagatgaagccattaaaaacaatgcctttaaaaaatgtcctttcgttcatccaaccgtgatgtttaaatcgtctgtatttaataacgggatgcgataccctgaaaatcagccactgacagaagatatgtgcttatggctcgagttaatatccgcgaataaaaagtttgcaaatattgactccgttcttctaaagtacagaattaatgagcagacgctaaaaagaagaagaggtttgaataaggctgtaaccgaattatccacaagattgtattacgccaagaaacttaaaatgcttagcgtacggaatttgttatatatattaatccattttggtgtcagagtccttccttcaaatatatttaaacttgtacataagttaaattaaaatgaataaaactgtttatacatatgatgtatgggatactattttaaaacgtcattgtttaccatcatatacgttagaggtttcgttgtactggctacttttgtatcttggaaaacctgagaaacataaggatgtgtataaaaaagtaaaagcactggagagtgatttttttaatcgaaaagaagaatattttattgaagagcttattgttgggatattaattgaaaataatttaatgaatggattttgcaataactctgtgactgctgcttgggaacaaatatattatttcgtagagagaaattgtacatataaaaataaagaggttatagagttaattgggagtgactatggggacaaattatttatttctgatttttatactgactccagtttcataaagaaattgctacaccaccaccaagtatcggttttagatggtgtgacgtcagctgagtatggcgtcagtaagcatatgggttcattgtatgaaaaaatagaactattgaagcaaaggaaatggatccatactggagataatgaattagctgatataaaaatggcaaagcgtgcaggtgcccaagttagacttataaaatccaaaaagaagaaaataaataagttaaaaataaaaaatgactatgagcggttagcatttattattttgtcctttagcctgtttatattaaaaacttccagaaagaaaaagtgcaataagatctacttttttacaagggagggggtttttttcaaaaaaaactttgatcttctactaaaccaaatacctaacatattcccatctataactacggaaatattgccaatcagtcgagtagccagcttagcattgaagtttaatgagtctgatgaatttttaggatttaatgatgcattaacccagtatggctatggagtgtctacgtttcttagtttctttcatcttgatgattgttactccgagctcacaacaaagtatcatgatgtaaatgatttattatctcacagaaatgatcctttggtaaaacaactaataaaaagtatagaggataaaaaaaaacgaacagaaaattttcttacatccattgagttcgattctgagccaagtatcatcgtggatattggttggcggggtagcatccaagataatttaatgtcgcggaaaaataattatattcaacatggatgttatctcggattgtttgatttttacccaggccagtcaggtcagaataaatcatctgtaatttttgataacaacagatttagacaaaaatggaccatgaaaggtgtagcttttatggagacggttttcaatgctttggacggaagtgttgtcgattatgtaaacaataaaccgaaaagaaaagaaaattacgctgagatagaaaattcaaaacatttaatagttatgcaagatgctatcattgaggaatttatgaatctatctattatgttaaaagagggaaagttaaatatcaatgatattgaaagattagctattgaaagttacaaaaaaatagtaactaacccatctaaagaaatagctgatttttatgttggcagtatccaaaatgaaagcttcggtttgaatgattttatcgacaaagaatttaacattagcgcagtagatgttttccgaagttttttttctaaaagtaaaagaaatgaaataagaataaaattgcatagaaatggatggcgtgagtcgatacttaaatcctctaaagtatcgctgagcgcaaaattatgttcacttttaattagataaaatatgaatatttcagtaataatttgttgctataactcagaaaatagaatcgtaaacaccctttttcatctttcacaatgtgtatctctatccaaggggttaattgacgagattattcttgttgacaataactcatctgatgaaactgttaaaacagctgaggatttttggcgtaatacgggggaggagcaaatctttaaaattgtttttgaatcgattccgggccttgttcatgctagacggcgagggctggacgagtccaaaaatgagattgttgttttttgtgatgatgataattggctggtaccatcttattttacaaaactagagaaatattttgaaaacaaacaagtaggcgctgtcgggggacgcggtattcctattgctaataccgttattcctgagtggttttttgagaatctggatgcatatgcatgttctgcacaaacttctaacatgttgtatggtgctagcttggctattcggtctagtatcttaaaaaaattttatacggatgctttatcagtaaagctaaagggtagaacaaaagacagtctggattctggggaagataacttattgtgtaattttgtcaaaaaacaaggatacactctgatacctgacaatacaccattttgccatttcatggaaagcaagcgattggatgtcgattatctatataaactggctgaatcatttggaaaagcaacggcatcaatgaaaaacaattccttcaccaccgattatgtatatttgacagcaaaaatgttattagctatatctaggatcttctctaaaaaaaataaaaagatatatttaaaacgtgagatttcctttctcagaggttatttgaggtttagaaaggaatcgaagtaatgagagggggaatgcttgtaattgcgtggtattctatttttctgttgatttcagctacgggcatgaccggtctaaataagctgagtactgaagctattctgtgctatattgcatttttattctgttttttctttggcatgatgttaggtggtgtttcgctcccaaacggaatgaaaatgttgcaacaggagcaaaagataaagaaaaggcatgcttctgccatagttctaatttattgcttttttattttttattctttaattgctcttttaaatatatattatttcaataaggttgatctatcatcttatagacaagcattttttgatggttcaagagataatgttaaatacttctatggcgtgtttggcttttacttctattatttacttagcctgttagtattcgccttagttccttatactattataacgaacagcaaaaagactatttcattatgtttctttgccttgctgctatacgatattattttcctttcaagaactggtgtttattactttatattatcttatatagtatcgagtattcttctcaagaaaggaatcaagaaaattataattgtagggatggttgccttattattctcattgataatgagttacacaagggatatgaagagtgacatcattggtacattatttagctcaattataaattatcatatagctccgtttgtcttgtttgatgaaaatataatatcaaatcatttgattaaatataatggggctggcttggcatcgttggggatatataatatcattttttatcccattgattcgtctatcatgtcatcaataaacgattttagagcacagttgaatgaattctatgatttgggagtaaatcgttatttgccatataatgcctattatacttctttgggtggggtatatatagatagcggcctgattggtagcatgatggttagttttatgactggttttattattgtcggaatcgagaagagtgcttttaaatcaactaagttcttagtctcttctgtgttttttactacattatgtatggaatctatattttccccaataacactaaatatttttgcctttacaataattgtctacttagttgttttgatggtccttaaatatgaaaatagcgatgataataccatctttaaaaaataccgcgccaaataatatagccctttcaatatgtgaaggggcaaggaagcatggtgtcaatatagatatttattatcttgatgaaattgtagagttggatatagacgagtcagaactgaatataaataggctaaatgatagaaccattttaaacagttatgatattgttcactctcatggcttaagacccgatgttattaattgtaagaccagcaaaaatgtcattaatgtaagtacgcttcatagttttattttagatgatcttaagaataaatatggaataaaaggatatctgatcgcttttgcttggcttaaattattaaagcgatttgattcatgtattgcaattagtgaggcgactaaacggttttatgttaaacatggtcttaaatgccaccatatatataatggaatcgactttcctgacatcgaattatatcaagataactcaaggctaaataacggcaaaataaatatagtatcgatcgcgcatctggaaaaaataaaaggcttagagcaactcctatatctagccgcggagcgagaagagttccatgttcatataattggtgaaggaacttaccgtgggaaattaacaaatattattgataagtttgatttatctcagcgtgtaactttgcatggatatatatcaaatgcgagttctatgttggggaaatttgatgtttatgtgcaaccatctaaaagtgaggggtttggcattgcagtcattgaagctcttgtaaataaaatcccaacagtttgctcagacattgaagtgttcagagagttatttggctcaggagaagttgaatttttcaagttagacgatattaattcactttacgatgcaatttcaagtgcattaaccaagacagatatgtctcgagatgcaagtgccacagcgataagccaaaaattttcttcagaagtgatgtcattgaactatttaagttggtataaaaaattatatgaagaaagaaatttataaatatggcacaataatattatgctatggaattaactttatactacctattcttttctttaaagaaataagtttaatattatctccaatagtattatcatcatttttctttgttttggcattgctaatgtattgtcaattcattgttgaatacgggttgcaaatatcattattaagaaagttaaatgaaaataagagtgatttaagtcggcttttatctactattcttggactaaaagtaatactattcgtcctatgcgcagtattcatttattgtattttgctatataataatgaagttatattttttatacttgttttcattctgctaggaaacgtattctcatgccagttcctctatcaagtagttgatcagctccattttttttatgtattaaattcgcttgttaagctaattttcatacccctcatttttatcaatgataattatatctatctgatgatatgttatagttgtttcaatatcctacctaacttagctgcacttgcttattttttatataagaataaagttaaggttgtaaaagttcctgcttgtgaaatttataatttatctaaggaatgttttagctattttatctcgaatatttcgataacattatacactaatttttatcagatcttattaggcattatatcgcccgcttatctggccgcgtatgcattaagtgacaaaattattagggggatcgtgtcagggaattatgtgtttatacagatagtacaagtacaatacttaaatgatgaatcaaagttcatcaatggtacatggaaaacgattgtttcattattattactgggggggatggagtttttatttattttctttggagcggattttttctctcgttatttattccctacaatatctttactgccaatttttctaaaactaatgtcattgctaattgttgttatcttgttgagtaacttttttgcaatggtttatcttccgtgttcaggcaatactttattcctctcacgtgttctgatttttgtttctattgtgtccgtagtagttgccccgatactaatatggctctatgcaggttatggtgcagtcatttccgctatattttcagagttattagttctgattttatgctatggtttatatttgaaattaaaacacaaactctaaggcgccacgcgtcctaaggttaattaagcggccgcgtcgactcgaggatccgtgtaggctggagctgcttcgaagttcctatactttctagagaataggaacttcggaataggaactaaggaggatattcatatggataaagccgtaagcatataagcatggataagctatttatactttaataagtactttgtatacttatttgcgaacattccaggccgcgagcattcagcgcggtgatcacacctgacaggagtatgtaatgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtaccggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcagaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtattgataaagaaggtgtgttccataccgaatggctggattaa

SEQ ID NO: 4 - protein, 713 aa, Campylobacter jejuni
  source 1..713: note = Example PglB sequence
MLKKEYLKNPYLVLFAMIILAYVFSVFCRFYWVWWASEFNEYFFNNQLMIISNDGYAFAEGARDMIAGFHQPNDLSYYGSSLSALTYWLYKITPFSFESIILYMSTFLSSLVVIPTILLANEYKRPLMGFVAALLASIANSYYNRTMSGYYDTDMLVIVLPMFILFFMVRMILKKDFFSLIALPLFIGIYLWWYPSSYTLNVALIGLFLIYTLIFHRKEKIFYIAVILSSLTLSNIAWFYQSAIIVILFALFALEQKRLNFMIIGILGSATLIFLILSGGVDPILYQLKFYIFRSDESANLTQGFMYFNVNQTIQEVENVDLSEFMRRISGSEIVFLFSLFGFVWLLRKHKSMIMALPILVLGFLALKGGLRFTIYSVPVMALGFGFLLSEFKAIMVKKYSQLTSNVCIVFATILTLAPVFIHIYNYKAPTVFSQNEASLLNQLKNIANREDYVVTWWDYGYPVRYYSDVKTLVDGGKHLGKDNFFPSFALSKDEQAAANMARLSVEYTEKSFYAPQNDILKTDILQAMMKDYNQSNVDLFLASLSKPDFKIDTPKTRDIYLYMPARMSLIFSTVASFSFINLDTGVLDKPFTFSTAYPLDVKNGEIYLSNGVVLSDDFRSFKIGDNVVSVNSIVEINSIKQGEYKITPIDDKAQFYIFYLKDSAIPYAQFILMDKTMFNSAYVQMFFLGNYDKNLFDLVINSRDAKVFKLKI

SEQ ID NO: 5 - protein, 535 aa, synthetic construct
  source 1..535: note = Example CRM197 sequence
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 6 - DNA, 9348 bp, synthetic construct
  source 1..9348: mol_type = other DNA; note = Rfb cluster O21
atgacgaatttaaaagcagttattcctgtagcgggtctcgggatgcatatgttgcctgccactaaggcgatacccaaagagatgctaccaatcgtcgacaagccaatgattcagtacattgttgacgagattgtggctgcagggatcaaagaaatcctcctggtaactcacgcgtccaagaacgcggtcgaaaaccacttcgacacctcttatgagttagaatcactccttgagcagcgcgtgaagcgtcaactgctggcggaagtacagtccatctgtccgccgggcgtgaccattatgaacgtgcgtcagggcgaacctttaggtttaggccactccattttgtgtgcgcgacctgccattggtgacaacccatttgtcgtggtactgccagacgttgtgatcgacgatgccagcgccgacccgctacgttacaaccttgctgccatgattgcacgtttcaacgaaacgggccgcagccaggtgctggcaaaacgtatgccgggtgacctctctgaatactccgtcatccagactaaagagccgctggaccgtgagggtaaagtcagccgcattgttgaatttatcgaaaaaccggatcagccgcagacgctggactcagacatcatggccgtaggtcgctatgtgctttctgccgatatttggccggaactggaacgtactcagcctggtgcatggggacgtattcagctgactgatgctattgccgagctggcgaaaaaacaatccgttgatgcaatgctgatgaccggcgacagttacgactgcggcaaaaaaatgggctatatgcaggcgtttgtgaagtatggcctacgcaacctgaaagaaggggcgaagttccgtaaaggtattgagaagctgttaagcgaataatgaaaatctgaccggatgtaacggttgataagaaaattataacggcagtgaaaattcgcagcaaaagtaatttgttgcgaatcttcctgccgttgttttatataaaccatcagaataacaacgagttagcagtagggttttattcaaagttttccaggattttccttgtttccagagcggattggtaagacaattagcgtttgaatttttcgggtttagcgcgagtgggtaacgctcgtcacatcataggcatgcatgcagtgctctggtagctgtaaagccaggggcggtagcgtgcattaatacctctattaatcaaactgagagccgcttatttcacagcatgctctgaagtaatatggaataaattaagctagaatgtttacaattaaaactgtatttgtgttgaggatattcggcgctatacttgcgctgtcaacgagtgtgatgatatcaagaacgctcaatatgagtgaggctggtggggttttcttcctattatcagtaactggaataatatcgtccctatcctctttaggacaaaataatttaattttgaaaaaatgtgcctcagttaaatatgcattaaaagcacgaggcgttttttttcttagatgcataaaacggagtgttatatccagtattatcttatctatcattttaatacctataattcattatttttcgccacaattattattacgaaatacatggtgtttggcgattctgttgataatatcatcgtcagtaaatatattgttatattcctttttacagtctcaaggtcttgtcacttttagtgttgttctgcagtatatatttcaacccttattgtttattttttttgtattacttattgttctgattaaaaatgaaagtgttttactggtgtctttatcatacttcttctcaattattcttattggaggaggggggggggtatactcgtattataaatttagggacgatttcaatcagttagaaacagagtcaataccgtttagactgaaagaattatcagagtatttcattggtaactcattaggaatgctgattgttcaatcttatgttgtactttccggtttgttgctcccagctgctgatgtcgctattattgctgtgtctgatagaatatccttggttataaatttatttgcaatgtcaattagcacaattctggcacctaaggtcgccagtttatacagccaaagtaaaatgaatgaaataaaagagctaactaaaaaagctatgttcttcataatgataccatgtgttttaatggctcttctgtttccctttttttcagggctgatattatctatatttggtgttcaatacagtaatgctagcgaagtgttaattattttagttatgacgcagattattaatgcaatattttgccctgtatatgtttttttaaatatgagtgatcggcaaggttttataagtaaattgcatatatatatgcttataccgtctcttgttataacattttatctgactaacatatttggtgttgttggaatagctgtgtctaagttaattgtcgtgtcattgattaatataatcccattgatttactgtattttattgtctaatcgagtgataaagtgatgagtttatattctattttaaaaaagttaattttatcttttcattactatctttttactatttcctttttatctgcgaaaaacgataagaagtactccaatcggttagattttgatgattatgttgcagctaagctaagaaaacagaatgaatataagaggaaaataccaagtgtctctgttatatatagagtcaaaaacggatctgaatatatagaggcatctatattgtctatttcggggttagcatcacaaataattgttgtagataataattctacagataatacaagagatattgtagaaagattatcgaatcaattagctgatgtttgctctatacaactttactcttatgataaaaatctcgctattgctggagaagggtataaagatcaggtgacaaagggagatggttcactggccgaattttataattttagtttttctttaggtgattgtgattatttaatgaaagtcgatgcccattatattttttcggcatacggtgttgatattttgcaaaagcagatagcaaaaaattatgatggtgtcgtttttcgagggttagagttttttgggaagtggatgagcaatgagctgtttttatataaacgttcactgggtctgaaatattgtgatggcgaattatatgaacagttaatgtgggataaatcaaccgtcagggtaaaaactatcattaaacccctttacttgcatgttaaacggttatcctatgttaaaaacctttataacacaaagaaagccatatacgtaaaatacaataaataggcaatagaatgaataataaaagaatatcaataataataccaacttataattcgagcataatattagaggaaaatattaaaataatcagtagctataagcagtgtaagatttttgtaattgatgatggttcagtttctgatgaagcgcgcaaaaataaaattatttgtaataattacgggtgtaattacacctatactcttaatcaaggcccttctcacgcaagatatgtaggcttattggattcagatacagaattttgtttttttcttgatactgatgattatattgctgaatttgctcttgattggggtgttaattatttattagaaaataatgaggttgatgccatcgtctttagaagtgattatgtggaagagtttaattgtaaaaacgagacaggtaatattttcacaataaaaaaatataacagaggattaatacaagaatgtatagagtgtttagggtatccaaagcaatttactcttggttggaatcaatctaatacgttgtatcgtagaaataaaatcattgatgcttataaaataagatatttgacttggggggaagatataccattaaaactaaaactaatttctgttatgtcgctaatttctgtgagaactctgggagggtctcaaattaaaatatcctatggacggggatataaatatacaccaaaacaaatagttgagcttgctctggaggtttacagaacgtccctaaataatgcattttgtcttgcattcataacagttataagatatatgctttcatatttttataaaagattaaaacaactgtccagtaaaaggaagtgagaatgaaaagaaaacttgttgacttttgtataatatcattacctcaacataatgaaaggagagataaattaaaaaatgaaatggcgaagtatgacatagaatgtcgtgtttctcatgctattgatggacggaaattattggcagaaaaatacttttctttatttaaaattaggtcctcaaagatgtttggaagaggttttttaactccgtcggaattgggatgtttcttgagtcataaaaaagctttaacggaatttttggcgagtggaaggaaatggttggtggttttggaagatgacgttttgcccaaggaaaatgttaaatatttggacgaaatgattaattcattctgctcatcaagtgtctatattttgggtggacaggatggtctaaaaagctttagtcgtgtcataatggggagaaaatcaatatgtggggtgcgaaaagtaatattaggtacacatcgttggttatataggacatgctgttattgtgtggatattaaaggcgctgagagaattctgaggttaatggaagaaaatagtttcttttgtgatgactggagttacattgtaagaaatgcgaaactggataatgtattttatggtcaatacttttctcatcctgtaaatttaaactctagttctatcgaagctgaacgtctttttattgcagaaaaatgatatatattataattaatactattgtgcttttttatatgtttttgtttaatgcatataatcgtattccttatgggaaattaattatcgggggcatatgcgttttttttatgactctgttgccagcatttcaatacggtgttggaacagattattttagctatcaaaatatatataataatgctaatgaactcgataccttttatcaaaataaggaatatctcttttatggatatatgaggctatatcaactgactggatggggattcaaaggttttattgcgttaactgctttattacaatctttattggtgtttgttattgtttttcagttatggaaaaactatggttattctcttgttttggttttttttcttttttgggttgttactaatttatttcatacccagatgaatataataagagcatcattttcaatttatttgtttgcaatatcaattctctataagtttagagggaaattattgttatctttcatattaatggttgctgctttaggttttcatcgctctgctttgattggtttctgttttctcgtaattccagataagatgtatttttttgcgtgtaaacatgcttttaaaatttatgtgttgacttttttattgtttcttttctcttatcttcagcaaattatttattatatagtgcaaaatttattcccctattactcacattatttatcctcattcgatgaaagttctgtgagtataatgaatgtactaactaaaatgtattggattccatttaatttgctattccttatcttattaagactaaaggtttttgttataaaagataatgagcgaaagttaattggtatgtgggctttaacggtgaatgtttatttgttaatgctcagttttgattttatttcaagggttaactactattttgtgatattttatataatcccgataatttatgttattaaatacactgttaagaatagatacttcgtttgtttgtatctctcattattatactgttttattccgtatctattaaaggttattctgtttcctatcgcagaattttattataaatcatacttgttctaattttgaataatgatggggctttttatgggtaatgaaacagtatcaataattatgccagcttataatgctgaagaaactattaaagatagtattttgtcaattttaaaacaaacgtatgaggattttaaattatatataatcaatgataattcgtcggattcaacagaacatattattaagtctattatcgatgaacgtattgtttatttactaaatcgaaatggtaagggagtatcttcggctagaaatgttgggattgctgcttgtaatggtagatatattgcattttgtgatagtgatgatgtttggtttgagactaaactcgaagaacaactgaaaatattgagtgctggaaattataaagttgtttgttcaaactatgaggttttttatgctgatacaaatgtaataaaagaaagaaggtttaaggaagttattacatataataatatgctgcaatctaatcatattggtaacttaactggtatttatgactcaactcagatagggaaagtatatcaacaggaaattggtcatgaagattacctaatgtggttaacaatagtaaaaaaagcaaagctggtttattgcatacagaaaaatcttgccagatattatatacataatacggggttgtcctcaaataagttcactgcagctatgtggcaatggaatatttatagaagagtattgtctttttcattattcaagtcattagttcttttttttatttattcagtaagagctcttgctaagagactttaaagatctaagcttaggaggtttcacctatgaacgataacgttttgctcataggagcttccggattcgtaggaacccgactacttgaaacggcaattgctgactttaatatcaagaacctggacaaacagcagagccacttttatccagaaatcacacagattggcgatgttcgtgatcaacaggcactcgaccaggcgttagccggttttgacactgttgtgctactggcagcggaacaccgcgatgacgtcagccctacttctctctattatgatgtcaatgttcagggtacccgcaatgtgctggcggccatggaaaaaaatggtgttaaaaatatcatctttaccagttccgttgctgtttatggtttgaacaaacacaaccctgacgaaaaccatccacacgaccctttcaaccactacggcaaaagcaagtggcaggcggaggaagtgctgcgtgaatggtataacaaagcaccaacagaacgttcattaactatcatccgtcctaccgttatcttcggtgaacgcaaccgcggtaacgtctataacttgctgaaacagatcgctggcggcaagtttatgatggtgggcgcagggactaactataagtccatggcttatgttggaaacattgttgagtttatcaagtacaaactgaagaatgttgccgcaggttatgaggtttataactacgttgataagccagacctgaacatgaaccagttggttgctgaagttgaacaaagcctgaacaaaaagatcccttctatgcacttgccttacccactaggaatgctgggtggatattgttttgatatcctgagcaaaattacgggcaaaaaatacgctgtcagctcagtgcgcgtgaaaaaattctgcgcaacaacacagtttgacgcaacgaaagtgcattcttcaggttttgtggcaccgtatacgctgtcgcaaggtctggatcgaactctgcagtatgaattcgttcatgccaaaaaagacgacataacgtttgtttctgagtaaacgcgtttaattaagcgatcgcactagtggatccgcggccgcgagctcgtcgactcgaggatccgtgtaggctggagctgcttcgaagttcctatactttctagagaataggaacttcggaataggaactaaggaggatattcatatggataaagccgtaagcatataagcatggataagctatttatactttaataagtactttgtatacttatttgcgaacattccaggccgcgagcattcagcgcggtgatcacacctgacaggagtatgtaatgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtaccggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcagaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtattgataaagaaggtgtgttccataccgaatggctggattaa

SEQ ID NO: 7 - DNA, 996 bp, synthetic construct
  source 1..996: mol_type = other DNA
atgaacgataacgttttgctcataggagcttccggattcgtaggaacccgactacttgaaacggcaattgctgactttaatatcaagaacctggacaaacagcagagccacttttatccagaaatcacacagattggcgatgttcgtgatcaacaggcactcgaccaggcgttagccggttttgacactgttgtgctactggcagcggaacaccgcgatgacgtcagccctacttctctctattatgatgtcaatgttcagggtacccgcaatgtgctggcggccatggaaaaaaatggtgttaaaaatatcatctttaccagttccgttgctgtttatggtttgaacaaacacaaccctgacgaaaaccatccacacgaccctttcaaccactacggcaaaagcaagtggcaggcggaggaagtgctgcgtgaatggtataacaaagcaccaacagaacgttcattaactatcatccgtcctaccgttatcttcggtgaacgcaaccgcggtaacgtctataacttgctgaaacagatcgctggcggcaagtttatgatggtgggcgcagggactaactataagtccatggcttatgttggaaacattgttgagtttatcaagtacaaactgaagaatgttgccgcaggttatgaggtttataactacgttgataagccagacctgaacatgaaccagttggttgctgaagttgaacaaagcctgaacaaaaagatcccttctatgcacttgccttacccactaggaatgctgggtggatattgttttgatatcctgagcaaaattacgggcaaaaaatacgctgtcagctcagtgcgcgtgaaaaaattctgcgcaacaacacagtttgacgcaacgaaagtgcattcttcaggttttgtggcaccgtatacgctgtcgcaaggtctggatcgaactctgcagtatgaattcgttcatgccaaaaaagacgacataacgtttgtttctgagtaa

SEQ ID NO: 8 - protein, 331 aa, synthetic construct
MNDNVLLIGASGFVGTRLLETAIADFNIKNLDKQQSHFYPEITQIGDVRDQQALDQALAGFDTVVLLAAEHRDDVSPTSLYYDVNVQGTRNVLAAMEKNGVKNIIFTSSVAVYGLNKHNPDENHPHDPFNHYGKSKWQAEEVLREWYNKAPTERSLTIIRPTVIFGERNRGNVYNLLKQIAGGKFMMVGAGTNYKSMAYVGNIVEFIKYKLKNVAAGYEVYNYVDKPDLNMNQLVAEVEQSLNKKIPSMHLPYPLGMLGGYCFDILSKITGKKYAVSSVRVKKFCATTQFDATKVHSSGFVAPYTLSQGLDRTLQYEFVHAKKDDITFVSE